SEQUENCE LISTING (67 sequences)

SEQ ID NO: 1 - DNA, 33 bp, Anaplasma marginale
acgaagtggcttctgaagggggagtaatgggag

SEQ ID NO: 2 - DNA, 30 bp, Anaplasma marginale
gactcacgcatgtcgaacgaggtaacagaa

SEQ ID NO: 3 - DNA, 30 bp, Anaplasma phagocytophilum
tgcggccgcagtatgtgcctgctccctttt

SEQ ID NO: 4 - DNA, 30 bp, Anaplasma phagocytophilum
gttataaccttttacgtaagatcctcccct

SEQ ID NO: 5 - DNA, 35 bp, Anaplasma ovis
agagacctcgtatgttagaggctatgacaagagtg

SEQ ID NO: 6 - DNA, 35 bp, Anaplasma ovis
ccttctgtagcttgcttctagttccactctagctc

SEQ ID NO: 7 - DNA, 30 bp, Artificial sequence
  GAPDH2_L primer
ctgagacaagatggtgaaggtcggagtgaa

SEQ ID NO: 8 - DNA, 27 bp, Artificial sequence
  GAPDH2_R primer
ttgccgtgggtggaatcatactggaac

SEQ ID NO: 9 - DNA, 38 bp, Artificial sequence
  Am3_probe
  misc_feature (1)..(1): 5′ Label -FAM
  misc_feature (23)..(23): THF
  misc_feature (38)..(38): 3′ block-C3 spacer
tagcttttacgtgggtgcggcctcagcccagcatttcc

SEQ ID NO: 10 - DNA, 30 bp, Artificial sequence; identical to SEQ ID NO: 2
  Am3R-nfo probe
  misc_feature (1)..(1): 5′Label biotin

SEQ ID NO: 11 - DNA, 51 bp, Artificial sequence
  Ap_probe
  misc_feature (1)..(1): 5′ Label-FAM
  misc_feature (33)..(33): THF
  misc_feature (51)..(51): 3′block - C3 spacer
tgatgcgtctgatgttagcggtgttatgaacggagcttttacgtaagtggt

SEQ ID NO: 12 - DNA, 30 bp, Artificial sequence; identical to SEQ ID NO: 4
  ApR_nfo probe
  misc_feature (1)..(1): 5′ Label Biotin

SEQ ID NO: 13 - DNA, 49 bp, Artificial sequence
  Ao2_probe
  misc_feature (1)..(1): 5′ Label-FAM
  misc_feature (32)..(32): THF
  misc_feature (49)..(49): 3′block-C3-spacer
aaatccggctacacttttgctttctctaagaattactcacatctttcga

SEQ ID NO: 14 - DNA, 35 bp, Artificial sequence; identical to SEQ ID NO: 6
  Ao2R-nfo probe
  misc_feature (1)..(1): 5′ Label-Biotin

SEQ ID NO: 15 - DNA, 50 bp, Artificial sequence
  GAPDH_probe
  misc_feature (1)..(1): 5′Label-DIG
  misc_feature (34)..(34): THF
  misc_feature (50)..(50): 3′block-C3spacer
ctggcaaagtggacatcgtcgccatcaatgacccttcattgaccttcact

SEQ ID NO: 16 - DNA, 27 bp, artificial sequence
  GAPDH_R_ nfo probe
  misc_feature (1)..(1): 5′Label-Biotin
ttgccgtgggtggaatcatactggaac

SEQ ID NO: 17 - DNA, 103 bp, Anaplasma marginale
acgaagtggcttctgaagggggagtaatgggaggtagcttttacgtgggtgcggcctacagcccagcatttccttctgttacctcgttcgacatgcgtgagtc

SEQ ID NO: 18 - DNA, 202 bp, Anaplasma phagocytophilum
tgcggccgcagtatgtgcctgctcccttttaattagtggatcctcatttgcgtattcaggcaataatgatgcgtctgatgttagcggtgttatgaacggcagcttttacgtaagtggtagctacagtccgtcatttccttctatatcttcatttgctattagtgagtctgacaggggaggatcttacgtaaaaggttataac

SEQ ID NO: 19 - DNA, 184 bp, Anaplasma ovis
agagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagccagagtggaactagaagcaagctacagaagg

SEQ ID NO: 20 - DNA, 168 bp, Bos taurus
ctgagacaagatggtgaaggtcggagtgaacggattcggccgcatcgggcgcctggtcaccagggctgcttttaattctggcaaagtggacatcgtcgccatcaatgaccccttcattgaccttcactacatggtctacatgttccagtatgattccacccacggcaa

SEQ ID NO: 21 - DNA, 69 bp, Anaplasma marginale
tagcttttacgtgggtgcggcctacagcccagcatttccttctgttacctcgttcgacatgcgtgagtc

SEQ ID NO: 22 - DNA, 135 bp, Anaplasma phagocytophilum
tgatgcgtctgatgttagcggtgttatgaacggcagcttttacgtaagtggtagctacagtccgtcatttccttctatatcttcatttgctattagtgagtctgacaggggaggatcttacgtaaaaggttataa

SEQ ID NO: 23 - DNA, 111 bp, Anaplasma ovis
aaatccggctacacttttgctttctctaagaatttactcacatctttcgacggcgctgtgggatattctctgggaggagccagagtggaactagaagcaagctacagaagg

SEQ ID NO: 24 - DNA, 91 bp, Bos taurus
ctggcaaagtggacatcgtcgccatcaatgaccccttcattgaccttcactacatggtctacatgttccagtatgattccacccacggcaa

SEQ ID NO: 25 - DNA, 852 bp, Anaplasma ovis
atgaattacagagaattgtttacagggggcctgtcagcagccacagtctgcgcctgctccctacttgttagtggggccgtagtggcgtctcccatgagtcatgaagtggcttctgaagggagcggggtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagccagagtggaactagaagcaagctacagaaggtttgctactttagcggacgggcagtacgcaaaaagtggtgcagagtccctggctgcaattactcgcgacgctgtcattactgagaacaattactttgtggtcaaaatcgatgaaatcacaaacacttcagtcatgctaaatggctgctatgacgtgttgcacacagatttgcctgtgtccccatatgtgtgtgccggaataggtgctagctttgtcgacatttctaagcaagtaaccacaaagctagcctacaggggcaaggttggaatcagctaccagtttactccagaaatatctttggtggtaggtgggttctaccacggactctttgacgagtcttacaaggacatacccgcacataacagtgtaaagttccctggagaagcaaaagcctcagtcaaggcacatattgctgattacggttttaaccttggagcaagattcctgttcagctaa

SEQ ID NO: 26 - DNA, 849 bp, Anaplasma marginale
atgaattacagagaattgtttacagggggcctgtcagcagccacagtctgcgcctgctccctacttgttagtggggccgtagtggcatctcccatgagtcacgaagtggcttctgaagggggagtaatgggaggtagcttttacgtgggtgcggcctacagcccagcatttccttctgttacctcgttcgacatgcgtgagtcaagcaaagagacctcatacgttagaggctatggcaagagcattgcaacgattgatgtgagtgtgccagcaaacttttccaaatctggctacaattttgccttctctaaaaacttaatcacgtctttcgacggcgctgtgggatattctctaggaggagccagagtggaattggaagcgagctacagaaggtttgctactttggcggacgggcagtacgcaaaaagtggtgcggaatctctggcagctattacccgcgacgctaacattactgagaccaattacttcgtagtcaaaattgatgaaatcacaaacacctcagtcatgttaaatggctgctatgacgtgctgcacacagatttgcctgtgtccccgtatgtatgtgccgggataggcgcaagctttgttgacatctctaagcaagtaaccacaaagctggcctacaggggcaaggttgggattagctaccagtttactccggaaatatccttggtggcaggtgggttctaccacgggctatttgatgagtcttacaaggacattcccgcacacaacagtgtaaagttctctggagaagcaaaagcctcagtcaaagcgcatattgctgactacggctttaaccttggagcaagattcctgttcagctaa

SEQ ID NO: 27 - DNA, 852 bp, Anaplasma ovis
atgaattacagagaattgtttacagggggcctgtcagcagccacagtctgcgcctgctccctacttgttagtggggccgtagtggcgtctcccatgagtcatgaagtggcttctgaagggagcggggtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagctagagtggaactagaagcaagctacagaaggtttgctactttagcggacgggcagtacgcaaaaagtggtgcagagtccctggctgcaattactcgcgacgctgtcattactgagaacaattactttgtggtcaaaatcgatgaaatcacaaacacttcagtcatgctaaatggctgctatgacgtgttgcacacagatttgcctgtgtccccatatgtgtgtgccggaataggtgctagctttgtcgacatttctaagcaagtaaccacaaagctagcctacaggggcaaggtaggaatcagctaccagtttactccagaaatatctttggtggtaggtgggttctaccacggactctttgacgagtcttacaaggacatacccgcacataacagtgtaacgttccccggagaagcaaaagcctcagtcaaggcccatattgctgattacggtgttgaccctggagcaagattcctgttcagctaa

SEQ ID NO: 28 - DNA, 852 bp, Anaplasma ovis
atgaattacagagaattgtttacagggggcctgtcagcagccacagtctgcgcctgctccctacttgttagtggggccgtagtggcgtctcccatgagtcatgaagtggcttctgaagggagcggggtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagctagagtggaactagaagcaagctacagaaggtttgctactttagcggacgggcagtacgcaaaaagtggtgcagagtccctggctgcaattactcgcgacgctgtcattactgagaacaattactttgtggtcaaaatcgatgaaatcacaaacacttcagtcatgctaaatggctgctatgacgtgttgcacacagatttgcctgtgtccccatatgtgtgtgccggaataggtgctagctttgtcgacatttctaagcaagtaaccacaaagctagcctacaggggcaaggttggaatcagctaccagtttactccagaaatatctttggtggtaggtgggttctaccacggactctttgacgagtcttacaaggacatacccgcacataacagtgtaaagttccccggagaagcaaaagcctcagtcaaggcacatattgctgattacggttttaaccctggagcaagattcctgttcagctaa

SEQ ID NO: 29 - DNA, 852 bp, Anaplasma ovis
atgaattacagagaattgtttacagggggcctgtcagcagccacagtctgcgcctgctccctacttgttagtggggccgtagtggcgtctcccatgagtcatgaagtggcttctgaagggagcggggtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagctagagtggaactagaagcaagctacagaaggtttgctactttagcggacgggcagtacgcaaaaagtggtgcagagtccctggctgcaattactcgcgacgctgtcattactgagaacaattactttgtggtcaaaatcgatgaaatcacaaacacttcagtcatgctaaatggctgctacgacgtgttgcacacagatttgcctgtgtccccatatgtgtgtgccggaataggtgctagctttgtcgacatttctaagcaagtaaccacaaagctagcctacaggggcaaggttggaatcagctaccagtttactccagaaatatctttggtggtaggtgggttctaccacggactctttgacgagtcttacaaggacatacccgcacataacagtgtaacgttccccggagaagcaaaagcctcagtcaaggcgcatattgctgattacggtttaaaccctggagcaagattcctgttcagctaa

SEQ ID NO: 30 - DNA, 852 bp, Anaplasma ovis
atgaattacagagaattgtttacagggggcctgtcagcagccacagtctgcgcctgctccctacttgttagtggggccgtagtggcgtctcccatgagtcatgaagtggcttctgaagggagcggggtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagctagagtggaactagaagcaagctacagaaggtttgctactttagcggacgggcagtacgcaaaaagtggtgcagagtccctggctgcaattactcgcgacgctgccattactgagaacaattactttgtggtcaaaatcgatgaaatcacaaacacttcagtcatgctaaatggctgctatgacgtgttgcacacagatttgcctgtgtccccatatgtgtgtgccggaataggtgctagctttgtcgacatttctaagcaagtaaccacaaagctagcctacaggggcaaggttggaatcagctaccagtttactccagaaatatctttggtggtaggtgggttctaccacggactctttgacgagtcttacaaggacatacccgcacataacagtgtaacgttccccggagaagcaaaagcctcagtcaaggcacatattgctgattacggtttgaaccctggagcaagattcctgttcagctaa

SEQ ID NO: 31 - DNA, 852 bp, Anaplasma ovis; identical to SEQ ID NO: 30

SEQ ID NO: 32 - DNA, 852 bp, Anaplasma ovis
atgaattacagagaattgtttacagggggcctgtcagcagccacagtctgcgactgctccctacttgttagtggtgccgtagtggcgtctcccatgagtcatgaagtggcttctgaagggagcggtgtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagctagagtggaactagaagcaagctacagaaggtttgctactttagcggacgggcagtacgcaaaaagtggtgcagagtccctggctgcaattactcgcgacgctgccattactgagaacaattactttgtggtcaaaatcgatgaaatcacaaacacttcagtcatgctaaatggctgctatgacgtgttgcacacagatttgcctgtgtccccatatgtgtgtgccggaataggtgctagctttgtcgacatttctaagcaagtaaccacaaagctagcctacaggggcaaggttggaatcagctaccagtttactccagaaatatctttggtggtaggtgggttctaccacggactctttgacgagtcttacaaggacatacccgcacataacagtgtaaagttccccggagaagcaaaagcgtcagtcaaggcgcatattgctgattacagtgttacccttggagcaagattcctgttcagctaa

SEQ ID NO: 33 - DNA, 852 bp, Anaplasma ovis
atgaattacagagaattgtttacagggggcctgtcagcagccacagtctgcgcctgctccctacttgttagtggtgccgtagtggcgtctcccatgagtcatgaagtggcttctgaagtgagcggtgtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagctagagtggaactagaagcaagctacagaaggtttgctactttagcggacgggcagtacgcaaaaagtggtgcagagtccctggctgcaattactcgcgacgctgccattactgagaacaattactttgtggtcaaaatcgatgaaatcacaaacacttcagtcatgctaaatggctgctatgacgtgttgcacacagatttgcctgtgtccccatatgtgtgtgccggaataggtgctagctttgtcgacatttctaagcaagtaaccacaaagctagcctacaggggcaaggttggaatcagctaccagtttactccagaaatatctttggtggtaggtgggttctaccacggactctttgacgagtcttacaaggacatacccgcacataacagtgtaacgttccccggagaagcaaaagcctcagtcaaggcacatattgctgattacggttttaaccctggagcaagattcctgttcagctaa

SEQ ID NO: 34 - DNA, 852 bp, Anaplasma ovis
atgaattacagagaattgtttacagggggcctgtcagcagccacagtctgcgcctgctccctacttgttagtggggccgtagtggcgtctcccatgagtcatgaagtggcttctgaagggagcggggtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagctagagtggaactagaagcaagctacagaaggtttgctactttagcggacgggcagtacgcaaaaagtggtgcagagtccctggctgcaattactcgcgacgctgtcattactgagaacaattactttgtggtcaaaatcgatgaaatcacaaacacttcagtcatgctaaatggctgctatgacgtgttgcacacagatttgcctgtgtccccatatgtgtgtgccggaataggtgctagctttgtcgacatttctaagcaagtaaccacaaagctagcctacaggggcaaggttggaatcagctaccagtttactccagaaatatcttcggtggtaggtgggttctaccacggactctttgacgagtctaacaaggacatacccgcacataacagtgtaacgttccccggagaagcaaaagcctcagtcaaggcacatattgctgattacggttgtgaccctggagcaagattcctgttcagctaa

SEQ ID NO: 35 - DNA, 852 bp, Anaplasma ovis
atgaattacagagaattgtttacagggggcctgtcagcagccacagtctgcgcctgctccctacttgttagtggggccgtagtggcgtctcccatgagtcatgaagtggcttctgaagggagcggggtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagctagagtggaactagaagcaagctacagaaggtttgctactttagcggacgggcagtacgcaaaaagtggtgcagagtccctggctgcaattactcgcgacgctgtcattactgagaacaattactttgtggtcaaaatcgatgaaatcacaaacacttcagtcatgctaaatggctgctatgacgtgttgcacacagatttgcctgtgtccccatatgtgtgtgccggaataggtgctagctttgtcgacatttctaagcaagtaaccacaaagctagcctacaggggcaaggttggaatcagctaccagtttactccagaaatatctttggtggtaggtgggttctaccacggactctttgacgagtcttacaaggacatacccgcacataacagtgtaacgtcccccggagaagcaaaagcctcagtcaaggcacatattgctgattacgggtttaaccctggagcaagattcctgttcagctaa

SEQ ID NO: 36 - DNA, 852 bp, Anaplasma ovis
atgaattacagagaattgtttacagggggcctgtcagcagccacagtctgcgcctgctccctacttgttagtggggccgtagtggcgtctcccatgagtcatgaagtggcttctgaagggagcggggtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagctagagtggaactagaagcaagctacagaaggtttgctactttagcggacgggcagtacgcaaaaagtggtgcagagtccctggctgcaattactcgcgacgctgccattactgagaacaattactttgtggtcaaaatcgatgaaatcacaaacacttcagtcatgctaaatggctgctatgacgtgttgcacacagatttgcctgtgtccccatatgtgtgtgccggaataggtgctagctttgtcgacatttctaagcaagtaaccacaaagctagcctacaggggcaaggttggaatcagctaccagtttactccagaaatatctttggtggtaggtgggttctaccacggactctttgacgagtcttacaaggacatacccgcacataacagtgtaaagttccccggagaagcaaaagcctcagtcaaggcacatattgctgattacggttttaccctgggagcaagattcctgttcagctaa

SEQ ID NO: 37 - DNA, 852 bp, Anaplasma ovis
atgaattacagagaattgtttacagggggcctgtcagcagccacagtctgcgcctgctccctacttgttagtggggccgtagtggcgtctcccatgagtcatgaagtggcttctgaagggagcggggtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagctagagtggaactagaagcaagctacagaaggtttgctactttagcggacgggcagtacgcaaaaagtggtgcagagtccctggctgcaattactcgcgacgctgtcattactgagaacaattactttgtggtcaaaatcgatgaaatcacaaacacttcagtcatgctaaatggctgctatgacgtgttgcacacagatttgcctgtgtccccatatgtgtgtgccggaataggtgctagctttgtcgacatttctaagcaagtaaccacaaagctagcctacaggggcaaggtaggaatcagctaccagtttactccagaaatatctttggtggtaggtgggttctaccacggactctttgacgagtcttacaaggacatacccgcacataacagtgtaacgttccccggagaagcaaaagcctcagtcaaggcacatattgctgattacggttttgaccctggagcaagattcctgttcagctaa

SEQ ID NO: 38 - DNA, 852 bp, Anaplasma ovis
atgaattacagagaattgtttacagggggcctgtcagcagccacagtctgcgcctgctccctacttgttagtggggccgtagtggcgtctcccatgagtcatgaagtggcttctgaagggagcggggtcatgggaggtagcttttatgtgagtgcggcttacagcccagcgtttccctctgttacctcattcgacatgcgtgagtcaagcagagagacctcgtatgttagaggctatgacaagagtgttgcaacaattgatgtgagtgcgccagcaaacttttccaaatccggctacacttttgctttctctaagaatttactcacatctttcgacggcgctgtgggatattctctgggaggagctagagtggaactagaagcaagctacagaaggtttgctactttagcggacgggcagtacgcaaaaagtggtgcagagtccctggctgcaattactcgcgacgctgccattactgagaacaattactttgtggtcaaaatcgatgaaatcacaaacacttcagtcatgctaaatggctgctatgacgtgttgcacacagatttgcctgtgtccccatatgtgtgtgccggaataggtgctagctttgtcgacatttctaagcaagtaaccacaaagctagcctacaggggcaaggtaggaatcagctaccagtttactccagaaatatctttggtggtaggtgggttctaccacggactctttgacgagtcttacaaggacatacccgcacataacagtgtaaagttccccggagaagcaaaagcctcagtcaaggcacatattgctgattacggttttaaccttggagcaagattcctgttcagctaa

SEQ ID NO: 39 - DNA, 849 bp, Anaplasma phagocytophilum
atgaattacagagaattgcttgtagggagcctatctgaggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcctgccagcttcacgcaacatgaaccctcattcaagtttgctaagagtttactaacatcttttgacggtgcaactggctatgctattggcggagctagagtggaagtcgaagttggatataagaagtttgaaacgcttgctgagagtgactacaagcatataggatcacataattttgttgccgtgggtcgtgatgcgacattaactccggacaacttctttgtaatgaaaatagacagcgtcaaagatatatctgtaatgcttaacgcttgttacgacgttatgcatactgacttgcctgtatctccttacatgtgtgctgggttaggggcgagctttattaatatcgccgaccacgttacaagcaagttggcttatagaggaaaggtaggcgtaagttacaaacttacccctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 40 - DNA, 849 bp, Anaplasma phagocytophilum
atgaattacagagaattgcttgtagggagcctatctgcggccgcagtatgtgcctgctcccttttaattagtggatcctcatttgcgtattcaggcaataatgatgcgtctgatgttagcggtgttatgaacggcagcttttacgtaagtggtagctacagtccgtcatttccttctatatcttcatttgctattagtgagtctgacaggggaggatcttacgtaaaaggttataacaagaatttgagcacgttgaatgtttcagatcctgccagcttcacgcaacatgatccctcattcaagttcgctaagagtttactaacatcttttgacggtgcaactggctatgctattggcggagctagagtggaagttgaagttggatataaaaagtttgaaacgctcgctgaaagtgactacaagcatgtagaatcacataattttgttgctgtgggtcgtgacgcgacattaactccagacaacttctttgtaatgaaaatagacagcgtcaaagatatatctgtaatgcttaacgcttgttacgacgttatgcatactgacttgcctgtatctccttacatgtgtgctgggttaggggcgagctttattaatatcgccgatcacgttacaagtaagttggcttatagaggaaaggtaggcgtaagttacaagcttactcctgaaatatctttaatagctggaggttcttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 41 - DNA, 849 bp, Anaplasma phagocytophilum
atgaattacagagaattgcttgtagggagcctatctgcggccgcagtatgtgcctgctcccttttaattagtggatcctcatttgcgtactcaggcaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttgagcacgttgaatgtttcagatcctgccagcttcacgcaacatgatccctcattcaagttcgctaagagtttactaacatcttttgacggtgcaactggctatgctattggcggagctagagtggaagttgaagttggatataaaaagtttgaaacgctcgctgaaagtgactacaagcatgtagaatcacataattttgttgctgtgggtcgtgacgcgacattaactccggacaacttctttgtaatgaaaatagacagcgtcaaagacatatctgtaatgcttaacgcttgttacgacgttatgcatactgacttgcctgtatctccttacatgtgtgctgggttaggggcgagttttattaatatcgccgatcacgttacaagtaagttggcttatagtggaaaggtaggcgtaagttacaagcttactcctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 42 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcctgccagcttcacgcaacatgatccctcattcaagttcgctaagagtttactaacatcttttgacggtgcaactggctatgctattggcggagctagagtggaagttgaagttggatataaaaagtttgaaacgctcgctgaaagtgactacaagcatgtagaatcacataattttgttgctgtgggtcgtgacgcgacattaactccggacaacttctttgtaatgaaaatagacagcgtcaaagacatatctgtaatgcttaacgcttgttacgacgttatgcatactgacttgcctgtatctccttacatgtgtgctgggttaggggcgagttttattaatatcgcagaccacgttacaagcaagttggcttatagaggaaaggtaggcgtaagttacaaacttacccctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 43 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcccgccagcttcacgcaacatgaaccctcattcaagtttgctaagagtttactaacatcttttgacggtgcaactggctatgctattggcggagctagagtggaagtcgaagttggatataagaagtttgaaacgcttgctgagagtgactacaagcatatagaatcacataattttgttgctgtgggtcgtgatgcgacattaactccggacaacttctttgtaatgaaaatagacagcgtcaaagatatatctgtaatgcttaacgcttgttacgacgttatgcatactgacttgcctgtatctccttacatgtgtgctgggttaggggcgagctttattaatatcgccgaccacgttacaagcaagttggcttatagaggaaaggtaggcgtaagttacaaacttacccctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 44 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcctgccagcttcacgcaacatgaaccctcattcaagttcgctaagagtttactaacatcttttgacggtgcaactggctatgctattggcggagctagagtggaagttgaagttggatataaaaagtttgaaacgctcgctgagagtgactacaagcatgtagaatcacataattttgttgctgtgggtcgtgacgcgacattaactccggacaacttctttgtaatgaaaatagacagcgtcaaagatatatctgtaatgcttaacgcttgttacgacgttatgcatactgacttgcctgtatctccttacatgtgtgctgggttaggggcgagctttattaatatcgccgaccacgttacaagcaagttggcttatagaggaaaggtaggcgtaagttacaaacttacccctgaaatatctttaatagctggaggtttttaccacggaattttcgacgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 45 - DNA, 849 bp, Anaplasma phagocytophilum; identical to SEQ ID NO: 44

SEQ ID NO: 46 - DNA, 849 bp, Anaplasma phagocytophilum
atgaattacagagaattgcttgtagggagcctatctgcggccgcagtatgtgcctgctcccttttaattagtggatcctcatttgcgtattcaggcaataatgatgcgtctgatgttagcggtgttatgaacggcagcttttacgtaagtggtagctacagtccgtcatttccttctatatcttcatttgctattagtgagtctgacaggggaggatcttacgtaaaaggttataacaagaatttgagcacgttgaatgtttcagatcctgccagcttcacgcaacatgatccctcattcaagttcgctaagagtttactaacatcttttgacggtgcaactggctatgctattggcggagctagagtggaagttgaagttggatataaaaagtttgaaacgctcgctgaaagtgactacaagcatgtagaatcacataattttgttgctgtgggtcgtgacgcgacattaactccagacaacttctttgtaatgaaaatagacagcgtcaaagatatatctgtaatgcttaacgcttgttacgacgttatgcatactgacttgcctgtatctccttacatgtgtgctgggttaggggcgagctttattaatatcgccgatcacgttacaagtaagttggcttatagtggaaaggtaggcgtaagttacaagcttactcctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 47 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcctgccagcttcacgcaacatgatccctcattcaagttcgctaagagtttactaacatcttttgacggtgcaactggctatgctattggcggagctagagtggaagttgaagttggatataaaaagtttgaaacgctcgctgaaagtgactacaagcatgtagaatcacataattttgttgctgtgggtcgtgacgcgacattaactccggacaacttctttgtaatgaaaatagacagcgttaaagacatatctgtaatgcttaacgcttgttacgacgttatgcatactgacttgcctgtatctccttacatgtgtgctgggttaggggcgagttttattaatatcgcagaccacgttacaagcaagttggcttatagaggaaaggtaggcgtaagttacaaacttacccctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacatacgagcaagatttgctttcaattaa

SEQ ID NO: 48 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctatagtccgtcatttccttctatatcttcatttgctattagtgagtccgacaggggaggatcttacgtaaaaggttataacaagaatttgagcacgttgaatgtttcagatcctgccagcttcacgcaacatgaaccctcattcaagtttgctaagagtttactaacatcttttgacggtgcaactggctatgctattggtggagctagagtggaagtcgaagttggatataagaagtttgaaacgcttgctgaaagcgactacaagcatgtagaatcacataattttgttgctgtgggtcgtgacgcgacattaactccggacaacttctttgtaatgaaaatagacagcgtcaaagatatatctgtaatgcttaacgcttgttacgacgttatgcatactgacttgcctgtatctccttacatgtgtgctgggttaggggcgagttttattaatatcgccgaccacgttacaagcaagttggcttatagaggaaaggtaggcgtaagttacaagcttactcctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 49 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcctgccagcttcacgcaacatgaaccctcattcaagtttgctaagagtttactaacatcttttgacggtgcaactggctatgctattggcggagctagagtggaagtcgaagttggatataagaagtttgaaacgcttgctgagagtgactacaagcatatagaatcacataattttgttgctgtgggtcgtgatgcgacattaactccggacaacttctttgtaatgaaaatagacagcgtcaaagatatatctgtaatgcttaacgcttgttacgacgttatgcatactgacttgcctgtatctccttacatgtgtgctgggttaggggcgagttttattaatatcgccgaccacgttacaagtaagttggcttatagaggaaaggtaggcgtaagttacaagcttactcctgaaatatctttaatagctggaggtttttaccacggaattttcgatgaacagtatgcaggtattcccgctagtaaccgtgtaaacatagcaggtggtgctgcagccaaggtaaaggcaaatatcgctagttacggcttcaacataggagcaagatttgctttcaattaa

SEQ ID NO: 50 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcctgccagcttcacgcaacatgatccctcattcaagttcgctaagagtttactaacatcttttgatggttcaactggctatgttattggcggagctagagtggaagttgaagttggatataaaaagtttgaaacgctcgctgaaagtgactacaagcatgtagaatcacataattttgttgctgtgggtcgtgacgcgacattaactccggacaacttctttgtaatgaaaatagacagcgttaaagacatatctgtaatgcttaacgcttgttacgacgttatgcatactgacttgcctgtatctccttacatgtgtgctgggttaggggcgagttttattaatatcgcagaccacgttacaagcaagttggcttatagaggaaaggtaggcgtaagttacaaacttacccctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 51 - DNA, 849 bp, Anaplasma phagocytophilum
atgaattacagagaattgcttgtagggagcctatctgcggccgcagtatgtgcctgctcccttttaattagtggatcctcatttgcgtactcaggcaataatgatgcgtctgatgttagcggtgttatgaacgggagcttttacgtgagtgggagctatagtccgtcattcccttctatatcttcatttgatattagtgagtccgacaggggaggatcttacgtaaaaggttataacaagaatttaagcacgttgaatgtttcagatcctgccagcttcacgcaacatgaaccctcattcaagtttgctaagagtttactaacatcttttgacggtgcaactggctatgctattggtggagctagagtggaagtcgaagttggatataagaagtttgaaacgcttgctgaaagcgactacaagcatgtagaatcacataattttgttgctgtgggtcgtgacgcgacattaactccggacaacttctttgtaatgaaaatagacagtgtcaaagatatatctgtaatgcttaacgcttgttacgacgttatgcatactgacttgcctgtatctccttacatgtgtgctgggttaggggcgagctttattaatatcgcagaccacgttacaagcaagttggcttatagaggaaaggtaggcgtaagttacaggcttactcctgaaatatctttaatagctggaggtttttaccacggaattttcgatgaacagtatgcaggtattcccgctagtaaccgtgtaaacatagcaggtggtgctgcagccaaggtaaaggcaaatatagctagttacggctttaacataggagcaagatttgctttcaattaa

SEQ ID NO: 52 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcctgccagcttcacgcaacatgaaccctcattcaagtttgctaagagtttactaacatcttttgacggtgcaactggctatgctattggcggagctagagtggaagttgaagttggatataaaaagtttgaaacgctcgctgaaagtgactacaagcatgtagaatcacataattttgttgctgtgggtcgtgacgcgacattaactccggacaacttctttgtaatgaaaatagacagtgtcaaagatatatctgtaatgcttaacgcttgttacgacgttatgcatactgacttgcctgtatctccttacatgtgtgctgggttaggggcgagctttattaatatcgcagaccacgttacaagcaagttggcttatagaggaaaggtaggcgtaagttacaggcttactcctgaaatatctttaatagctggaggtttttaccacggaattttcgatgaacagtatgcaggtattcccgctagtaaccgtgtaaacatagcaggtggtgctgcagccaaggtaaaggcaaatatagctagttacggctttaacataggagcaagatttgctttcaattaa

SEQ ID NO: 53 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttactattagtgagtctgacaggggaggatcttacgtaaaaggttataacaagaatttaagcacgttgaatgtttcagatcctgccagcttcacgcaacatgaaccctcattcaagtttgctaagagtttactaacatcttttgacggtgcaactggctatgctattggtggagctagagtggaagtcgaagttggatataagaagtttgaaacgcttgctgaaagcgactacaagcatgtagaatcacataattttgttgctgtgggtcgtgatgcgacattaactccggacaacttctttgtaatgaaaatagacagcgtcaaagatatatctgtaatgcttaacgcttgttacgacgttatgcatactgacttgcctgtatctccttacatgtgtgctgggttaggggcgagttttattaatatcgccgaccacgttacaagtaagttggcttatagaggaaaggtaggcgtaagttacaagcttactcctgaaatatctttaatagctggaggtttttaccacggaattttcgatgaacagtatgcaggcattcccgctagtaaccgtgtaaacatagcaggtggtgctgcagccaaggtaaaggcaaatatcgctagttacggcttcaacataggagcaagatttgctttcaattaa

SEQ ID NO: 54 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcctgccagcttcacgcaacatgatccctcattcaagttcgctaagagtttactaacatcttttgacggtgcaactggctatgctattggcggagctagagtggaagttgaagttggatataaaaagtttgaaacgctcgctgaaagtgactacaagcatgtagaatcacataattttgttgctgtgggtcgtgacgcgacattaactccggacaacttctttgtaatgaaaatagacagcgttaaagacatatctgtaatgcttaacgcttgttacgacgttatgcatactgacttgcctgtatctccttacatgtgtgctgggttaggggcgagttttattaatatcgcagaccacgttacaagcaagttggcttatagaggaaaggtaggcgtaagttacaaacttacccctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 55 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctatagtccgtcatttccttctatatcttcatttgctattagtgagtctgacaggggaggatcttacgtaaaaggttataacaagaatttaagcacgttgaatgtttcagatcctgccagcttcacgcaacatgaaccctcattcaagtttgctaagagtttactaacatcttttgacggtgcaactggctatgctattggtggagctagagtggaagtcgaagttggatataagaagtttgaaacgcttgctgaaagcgactacaagcatgtagaatcacataattttgttgctgtgggtcgtgacgcgacattaactccagacaacttctttgtgatgaaaatagacagcgtcaaagatatatctgtaatgcttaacgcttgttacgacgttatgcatactgacttgcctgtatctccttacatgtgtgctgggttaggggcgagttttattaacatcgccgaccacgttacaagcaagttggcttatagaggaaaggtaggcgtaagttacaagcttactcctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 56 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttactattagtgagtctgacaggggaggatcttacgtaaaaggttataacaagaatttaagcacgttgaatgtttcagatcctgccagcttcacgcaacatgaaccctcattcaagtttgctaagagtttactaacatcttttgacggtgcaactggctatgctattggtggagctagagtggaagtcgaagttggatataagaagtttgaaacgcttgctgaaagcgactacaagcatgtagaatcacataattttgttgctgtgggtcgtgacgcgacattaactccagacaacttctttgtgatgaaaatagacagcgtcaaagatatatctgtaatgcttaacgcttgttacgacgttatgcatactgacttgcctgtatctccttacatgtgtgctgggttaggggcgagttttattaacatcgccgaccacgttacaagcaagttggcttatagaggaaaggtaggcgtaagttacaagcttactcctgaaatatctttaatagctggaggtttttaccacggaattttcgatgaacagtatgcaggtattcccgctagtaaccgtgtaaacatagcaggtggtgctgcagccaaggtaaaggcaaatatcgctagttacggcttcaacataggagcaagatttgctttcaattaa

SEQ ID NO: 57 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcctgccagcttcacgcaacatgatccctcattcaagttcgctaagagtttactaacatcttttgacggtgcaactggctatgctattggcggagctagagtggaagttgaagttggatataaaaagtttgaaacgctcgctgaaagtgactacaagcatgtagaatcacataattttgttgccgtgggtcgtgacgcgacattaactccggacaacttctttgtaatgaaaatagacagcgtcaaagatatatctgtaatgcttaacgcttgttacgacgttatgcatactgacttgcctgtatctccttacatgtgtgctgggttaggggcgagctttattaatatcgccgaccacgttacaagcaagttggcttatagaggaaaggtaggcgtaagttacaaacttacccctgaaatatctttaatagctggaggtttttaccacggaattttcgacgaacagtatgcaggtattcccgctagtaaccgtgtaaacatagcaggtggtgctgcagccaaggtaaaggcaaatatcgctagttacggcttcaacataggagcaagatttgctttcaattaa

SEQ ID NO: 58 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcctgccagcttcacgcaacatgatccctcattcaagttcgctaagagtttactaacatcttttgacggtgcaactggctatgctattggcggagctagagtggaagttgaagttggatataaaaagtttgaaacgctcgctgaaagtgactacaagcatgtagaatcacataattttgttgccgtgggtcgtgacgcgacattaactccggacaacttctttgtaatgaaaatagacagcgttaaagacatatctgtaatgcttaacgcttgttacgacgttatgcatactgacttgcctgtatctccttacatgtgtgctgggttaggggcgagctttattaatatcgccgaccacgttacaagcaagttggcttatagaggaaaggtaggcgtaagttacaaacttacccctgaaatatctttaatagctggaggtttttaccacggaattttcgatgaacagtatgcaggtattcccgctagtaaccgtgtaaacatagcaggtggtgctgcagccaaggtaaaggcaaatatcgctagttacggcttcaacataggagcaagatttgctttcaattaa

SEQ ID NO: 59 - DNA, 849 bp, Anaplasma phagocytophilum
atgaattacagagaattgcttgtagggagcctatctgcggccgcagtatgtgcctgctcccttttaattagtggatcctcatttgcgtactcaggtaataatgatgcgtctgatgttagcggtgttatgaacgggagcttttacgtaagtggtagctacagtccgtcatttccttctatatcttcatttgctattagtgagtctgacaggggaggatcttacgtaaaaggttataacaagaatttaagcacgttgaatgtttcagatcctgccagcttcacgcaacatgaaccctcattcaagtttgctaagagtttactaacatcttttgacggtgcaactggctatgctattggcggagctagagtggaagtcgaagttggatataagaagtttgaaacgcttgctgagagtgactacaagcatatagaatcacataattttgttgctgtgggtcgtgatgcgacattaactccggacaacttctttgtaatgaaaatagacagcgtcaaagatatatctgtaatgcttaacgcttgttacgacgttatgcatactgacttgcctgtatctccttacatgtgtgctgggttaggggcgagctttattaatatcgccgaccacgttacaagcaagttggcttatagaggaaaggtaggcgtaagttacaaacttacccctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 60 - DNA, 849 bp, Anaplasma phagocytophilum
atgaattacagagaattgcttgtagggagcctatctgcggccgcagtatgtgcctgctcccttttaattagtggatcctcatttgcgtactcaggcaataatgatgcgtctgatgttagcggtgttatgaacggcagcttttacgtaagtggtagctacagtccgtcattcccttctatatcttcatttgatattagtgagtctgacaggggtggatcttacgtaaaaggttataacaagaatttgagcacgttgaatgtttcagatcctgccagcttcacgcaacatgatccctcattcaagttcgctaagagtttactaacatcttttgacggtgcaactggctatgctattggcggagctagagtggaagttgaagttggatataaaaagtttgaaacgctcgctgaaagtgactacaagcatgtagaatcacataattttgttgctgtgggtcgtgacgcgatattaactccagacaacttctttgtaatgaaaatagacagcgtcaaagatatatctgtaatgcttaacgcttgttacgacgttatgcatactgacttgcctgtatctccttacatgtgtgctgggttaggggcgagctttattaatatcgccgaccacgttacaagtaagttggcttatagaggaaaggtaggcgtaagttacaagcttactcctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 61 - DNA, 849 bp, Anaplasma phagocytophilum; identical to SEQ ID NO: 46

SEQ ID NO: 62 - DNA, 849 bp, Anaplasma phagocytophilum
atgaattacagagaattgcttgtagggagcctatctgcggccgcagtatgtgcctgctcccttttaattagtggatcctcatttgcgtattcaggcaataatgatgcgtctgatgttagcggtgttatgaacggcagcttttacgtaagtggtagctacagtccgtcatttccttctatatcttcatttgctattagtgagtctgacaggggaggatcttacgtaaaaggttataacaagaatttgagcacgttgaatgtttcagatcctgccagcttcacgcaacatgatccctcattcaagttcgctaagagtttactaacatcttttgacggtgcaactggctatgctattggcggagctagagtggaagttgaagttggatataaaaagtttgaaacgctcgctgaaagtgactacaagcatgtagaatcacataattttgttgctgtgggtcgtgacgcgacattaactccagacaacttctttgtaatgaaaatagacagcgtcaaagatatatctgtaatgcttaacgcttgttacgacgttatgcatactgacttgcctgtatctccttacatgtgtgctgggttaggggcgagctttattaatatcgccgatcacgttacaagtaagttggcttatagaggaaaggtaggcgtaagttacaagcttactcctgaaatgtctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 63 - DNA, 849 bp, Anaplasma phagocytophilum
atgaattacagagaattgcttgtagggagcctatctgcggccgcagtatgtgcctgctcccttttaattagtggatcctcatttgcgtattcaggcaataatgatgcgtctgatgttagcggtgttatgaacggcagcttttacgtaagtggtagctacagtccgtcatttccttctatatcttcatttgctattagtgagtctgacaggggaggatcttacgtaaaaggttataacaagaatttgagcacgttgaatgtttcagatcctgccagcttcacgcaacatgatccctcattcaagttcgctaagagtttactaacatcttttgacggtgcaactggctatgctattggcggagctagagtggaagttgaagttggatataaaaagtttgaaacgctcgctgaaagtgactacaagcatgtagaatcacataattttgttgctgtgggtcgtgacgcgacattaactccagacaacttctttgtaatgaaaatagacagcgtcaaagatatatctgtaatgcttaacgcttgttacgacgttatgcatactgacttgcctgtatctccttacatgtgtgctgggttaggggcgagctttattaatatcgccgatcacgttacaagtaagttggcttatagaggaaaggtaggcgtaagttacaagcttactcctgaaatatctttaatagctggaggtttttaccacggaattttcgatgaacagtatgcaggtattcccgctagtaaccgtgtaaacatagcaggtggtgctgcagccaaggtaaaggcaaatatagctagttacggcttcaacataggagcaagatttgctttcaattaa

SEQ ID NO: 64 - DNA, 849 bp, Anaplasma phagocytophilum; identical to SEQ ID NO: 40

SEQ ID NO: 65 - DNA, 1279 bp, Bos taurus
gctctctgctcctgcccgttcgacagatagccgtaacttctgtgctgtgccagccgcatccctgagacaagatggtgaaggtcggagtgaacggattcggccgcatcgggcgcctggtcaccagggctgcttttaattctggcaaagtggacatcgtcgccatcaatgaccccttcattgaccttcactacatggtctacatgttccagtatgattccacccacggcaagttcaacggcacagtcaaggcagagaacgggaagctcgtcatcaatggaaaggccatcaccatcttccaggagcgagatcctgccaacatcaagtggggtgatgctggtgctgagtatgtggtggagtccactggggtcttcactaccatggagaaggctggggctcacttgaagggtggcgccaagagggtcatcatctctgcaccttctgccgatgcccccatgtttgtgatgggcgtgaaccacgagaagtataacaacaccctcaagattgtcagcaatgcctcctgcaccaccaactgcttggcccccctggccaaggtcatccatgaccactttggcatcgtggagggacttatgaccactgtccacgccatcactgccacccagaagactgtggatggcccctccgggaagctgtggcgtgacggccgaggggctgcccagaatatcatccctgcttctactggcgctgccaaggccgtgggcaaggtcatccctgagctcaacgggaagctcactggcatggccttccgcgtccccactcccaacgtgtctgttgtggatctgacctgccgcctggagaaacctgccaagtatgatgagatcaagaaggtggtgaagcaggcgtcagagggccctctcaagggcattctaggctacactgaggaccaggttgtctcctgcgacttcaacagcgacactcactcttctaccttcgatgctggggctggcattgccctcaacgaccactttgtcaagctcatttcctggtacgacaatgaatttggctacagcaacagggtggtggacctcatggtccacatggcctccaaggagtaaggtccctggacccccagccccagcaggagcacgagaggaagagttcctcagctgctggggagtcctgccccatctccgccacactgagaatctcctgacttccacatgtttccatctctaaggccctgaggaaggggaggggcttagggagccctgccttgtgtaccatcaataaaagtaccctatacctaaaaaaaaaaaaaaaa

SEQ ID NO: 66 - DNA, 1280 bp, Bos taurus
ggctctctgctcctgcccgttcgacagatagccgtaacttctgtgctgtgccagccgcatccctgagacaagatggtgaaggtcggagtgaacggattcggccgcatcgggcgcctggtcaccagggctgcttttaattctggcaaagtggacatcgtcgccatcaatgaccccttcattgaccttcactacatggtctacatgttccagtatgattccacccacggcaagttcaacggcacagtcaaggcagagaacgggaagctcgtcatcaatggaaaggccatcaccatcttccaggagcgagatcctgccaacatcaagtggggtgatgctggtgctgagtatgtggtggagtccactggggtcttcactaccatggagaaggctggggctcacttgaagggtggcgccaagagggtcatcatctctgcaccttctgccgatgcccccatgtttgtgatgggcgtgaaccacgagaagtataacaacaccctcaagattgtcagcaatgcctcctgcaccaccaactgcttggcccccctggccaaggtcatccatgaccactttggcatcgtggagggacttatgaccactgtccacgccatcactgccacccagaagactgtggatggcccctccgggaagctgtggcgtgacggccgaggggctgcccagaatatcatccctgcttctactggcgctgccaaggccgtgggcaaggtcatccctgagctcaacgggaagctcactggcatggccttccgcgtccccactcccaacgtgtctgttgtggatctgacctgccgcctggagaaacctgccaagtatgatgagatcaagaaggtggtgaagcaggcgtcagagggccctctcaagggcattctaggctacactgaggaccaggttgtctcctgcgacttcaacagcgacactcactcttctaccttcgatgctggggctggcattgccctcaacgaccactttgtcaagctcatttcctggtacgacaatgaatttggctacagcaacagggtggtggacctcatggtccacatggcctccaaggagtaaggtccctggacccccagccccagcaggagcacgagaggaagagttcctcagctgctggggagtcctgccccatctccgccacactgagaatctcctgacttccacatgtttccatctctaaggccctgaggaaggggaggggcttagggagccctgccttgtgtaccatcaataaaagtaccctatacctaaaaaaaaaaaaaaaa

SEQ ID NO: 67 - DNA, 1289 bp, Bos taurus
attcgggctgcagcctcctcctgcgctctctgctcctgcccgttcgacagatagccgtaacttctgtgctgtgccagccgcatccctgagacaagatggtgaaggtcggagtgaacggattcggccgcatcgggcgcctggtcaccagggctgcttttaattctggcaaagtggacatcgtcgccatcaatgaccccttcattgaccttcactacatggtctacatgttccagtatgattccacccacggcaagttcaacggcacagtcaaggcagagaacgggaagctcgtcatcaatggaaaggccatcaccatcttccaggagcgagatcctgccaacatcaagtggggtgatgctggtgctgagtatgtggtggagtccactggggtcttcactaccatggagaaggctggggctcacttgaagggtggcgccaagagggtcatcatctctgcaccttctgccgatgcccccatgtttgtgatgggcgtgaaccacgagaagtataacaacaccctcaagattgtcagcaatgcctcctgcaccaccaactgcttggcccccctggccaaggtcatccatgaccactttggcatcgtggagggacttatgaccactgtccacgccatcactgccacccagaagactgtggatggcccctccgggaagctgtggcgtgacggccgaggggctgcccagaatatcatccctgcttctactggcgctgccaaggccgtgggcaaggtcatccctgagctcaacgggaagctcactggcatggccttccgcgtccccactcccaacgtgtctgttgtggatctgacctgccgcctggagaaacctgccaagtatgatgagatcaagaaggtggtgaagcaggcgtcagagggccctctcaagggcattctaggctacactgaggaccaggttgtctcctgcgacttcaacagcgacactcactcttctaccttcgatgctggggctggcattgccctcaacgaccactttgtcaagctcatttcctggtacgacaatgaatttggctacagcaacagggtggtggacctcatggtccacatggcctccaaggagtaaggtccctggacccccagccccagcaggagcacgagaggaagagttcctcagctgctggggagtcctgccccatctccgccacactgagaatctcctgacttccacatgtttccatctctaaggccctgaggaaggggaggggcttagggagccctgccttgtgtaccatcaataaaagtaccctatacccag

(19) United States
(12) Patent Application Publication
(10) Pub. No.: US 2021/0130878 A1
(43) Pub. Date: May 6, 2021

(54) COMPOSITIONS AND KITS FOR RAPID DETECTION SCREENING OF MULTIPLE ANAPLASMA SPECIES AND METHODS OF PRODUCTION AND USE THEREOF

(71) Applicant: The Board of Regents for the Oklahoma Agricultural and Mechanical Colleges, Stillwater, OK (US)

(72) Inventors: Bruce Noden, Stillwater, OK (US); Francisco Manuel OCHOA CORONA, Stillwater, OK (US)

(21) Appl. No.: 17/086,619

(22) Filed: Nov. 2, 2020

Related U.S. Application Data
(60) Provisional application No. 62/929,230, filed on Nov. 1, 2019.

(51) Int. Cl. C12Q 1/6825; C12Q 1/6841

(52) U.S. Cl. CPC C12Q 1/6825; C12Q 1/6841

(57) ABSTRACT

Kits, devices, systems, and methods are disclosed for use in recombinase polymerase amplification (RPA) assays for the detection of Anaplasma infections. In certain non-limiting embodiments, the assays can identify and discriminate between three Anaplasma species.

### CROSS REFERENCE TO RELATED APPLICATIONS/INCORPORATION BY REFERENCE STATEMENT

The subject application claims benefit under 35 USC § 119(e) and 35 USC § 21 of provisional application U.S. Ser. No. 62/929,230, filed Nov. 1, 2019. The entire contents of the above-referenced patent(s)/patent application(s) are hereby expressly incorporated herein by reference.

### STATEMENT REGARDING FEDERALLY SPONSORED RESEARCH OR DEVELOPMENT

This invention was made with U.S. Government support under Agriculture and Food Research Initiative Competitive Grant No. 2018-67016-28311 awarded by the USDA National Institute of Food and Agriculture. The Government has certain rights in this invention.

### BACKGROUND

Cattle production occupies a large part of the United States agricultural sector, generating approximately $50.2 billion. Cattle are raised in each state; however, the concentration increases in the central United States.

Ticks are blood-sucking arthropods that transmit a wide variety of pathogens like viruses, bacteria, and protozoa. Anaplasmosis is an important disease that is caused by the bacterium genus Anaplasma spp. and infects a broad range of animals such as (but not limited to) cattle, sheep, and humans. The three main species of concern are: A. marginale, A. ovis, and A. phagocytophilum. Detection methods for this pathogen include a wide variety of microscopy, antibody-based, and molecular methods. Currently, A. marginale detection in cattle uses a USDA-approved cELISA, a serologic test that targets the highly conserved Anaplasma msp5 gene; however, this test does not distinguish among species (De Waal, 2000; Atif, 2015). In addition, polymerase chain reaction (PCR) detects Anaplasma spp., but low diagnostic sensitivity for early and chronic infections, the expense of the equipment needed to run the assay, and the need for trained personnel limits its effectiveness in field conditions (Aubry and Geale, 2011).

LAMP and RPA are isothermal methods that overcome cost, time, and sensitivity issues encountered by traditional DNA-based diagnostics (Pai et al., 2012). LAMP assays have been developed to detect A. ovis, A. phagocytophilum, and A. marginale (Ma et al., 2011; Pan et al., 2011; Lee et al., 2012; Wen et al., 2016; Giglioti et al., 2019); however, to date, all of these assays are only being used in a laboratory-based setting.

Recombinase polymerase amplification (RPA) was developed in 2006 and represents an innovative isothermal amplification that has been used to detect a range of pathogens in agriculture and human and veterinary medicine, as well as food safety (James and Macdonald, 2015; Daher et al., 2016). The advantage of RPA is the minimal requirement for equipment due to low reaction temperatures (25-45° C.), relatively short incubation periods (20-40 min), and use of sensitive and specific primers and probe (Daher et al., 2016). Additionally, RPA reagents can be stored for at least 3 weeks at room temperature and without the need for low-temperature storage, because they are stable as lyophilized pellets (Lillis et al., 2016). Therefore, RPA can be applied directly in point-of-care diagnostic centers or resource-limited areas, while PCR or ELISA analyses require expensive laboratory equipment and trained personnel.

Given the complex nature of Anaplasma infections in livestock worldwide and the current lack of field-based diagnostic tools, a specific, sensitive, easy-to-use, and rapid isothermal detection method is needed to improve the accuracy of Anaplasma diagnosis. It is to such new and improved methods, as well as compositions, kits, and devices utilized in same, that the present disclosure is directed.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1. Flowchart showing the basic steps followed toward two nfo RPA assays constructed in accordance with the present disclosure.

FIG. 2. Basic RPA artificial positive control (APC) carrying primer sequences for A. marginale, A. ovis, A. phagocytophilum, and GAPDH housekeeping gene (internal control). Custom synthesized in tandem of RPA forward and reverse complement primer sequences ligated into a multiple cloning site of pUC57 vector.

FIG. 3. Lateral flow RPA artificial positive control (APC) carrying primer and probe sequences for A. marginale, A. ovis, A. phagocytophilum, and GAPDH housekeeping gene (internal control). Custom synthesized in tandem of RPA forward primer, reverse complement primer, and nfo probe sequences ligated into a multiple cloning site of pUC57 vector.

FIG. 4. RPA reactions A) Basic RPA APC and B) Lateral flow RPA. Lane L, 100 bp DNA ladder; lanes APC, artificial positive control; lane Am, A. marginale sample; lane Ao, A. ovis sample; lane Ap, A. phagocytophilum sample; lane IC, cattle blood sample, lane N, non-template control (water).

FIG. 5. Anaplasma spp. basic RPA in 2% gel electrophoresis in 1×TAE buffer. A) Basic RPA products with no purification step after RPA reaction, B) Basic RPA products with purification step after RPA reaction. Lane L, 100 bp DNA ladder; lane Ao, A. ovis; lane Ap, A. phagocytophilum; lane Am, A. marginale; lane N, non-template control (NTC, water).

FIG. 6. Temperature gradient of basic RPA for Anaplasma spp. Temperature ranged from 35 to 40° C.; lane N, non-template control (NTC, water); lane L, 100 bp DNA ladder.

FIG. 7. Anaplasma spp. detecting by nfo RPA using PCRD lateral flow devices. Lane C is the flow-check line; Lane 2 is the FAM/Biotin labelled amplicons; Lane 1, is intended for detection using DIG/Biotin labelled amplicons not used in this assay; Am is A. marginale; Ao is A. ovis; Ap is A. phagocytophilum; NTC is non-template control (water).

FIG. 8. A. marginale nfo RPA using lateral flow device. Lane C, flow-check line; Lane 2, detects FAM/Biotin labelled amplicons; Lane 1, is intended for detection using DIG/Biotin labelled amplicons not used in this assay; Am samples, A. marginale; NTC, non-template control (water).

FIG. 9. Specificity assay among Anaplasma spp. of basic RPA primers. Lane L, 100 bp DNA ladder; lane Am, A. marginale; lane Ao, A. ovis; lane Ap, A. phagocytophilum; lane T, lab-reared tick DNA; lane N, non-template control (NTC, water). Top gel is RPA reaction with A. marginale primers, center gel is RPA reaction with A. ovis primers and bottom gel is RPA reaction with A. phagocytophilum primers.

FIG. 10. Specificity assay among Anaplasma spp. of lateral flow RPA primers and probe. Lane C, flow-check line; Lane 2, detects FAM/Biotin labelled amplicons (Anaplasma species); Lane 1, detects DIG/Biotin labelled amplicons (GAPDH—internal control); Am, A. marginale; Ao, A. ovis; Ap, A. phagocytophilum; T, lab-reared tick DNA; N, non-template control (NTC, water).

FIG. 11. Sensitivity assays of A. marginale basic RPA primers. A) Ten-fold serial dilution of A. marginale plasmid from 1 ng/μl to 1 fg/μl. B) Ten-fold serial dilution of A. marginale total DNA from 0.01 ng/μl to 1 fg/μl. Lane N, non-template control (NTC, water); lane L, 100 bp DNA ladder.

FIG. 12. Sensitivity assays of A. ovis basic RPA primers. A) Ten-fold serial dilution of A. ovis plasmid from 1 ng/μl to 1 fg/μl. B) Ten-fold serial dilution of A. ovis total DNA from 1 pg/μl to 1 fg/μl. Lane N, non-template control (NTC, water); lane L, 100 bp DNA ladder.

FIG. 13. Sensitivity assays of A. phagocytophilum basic RPA primers. A) Ten-fold serial dilution of A. phagocytophilum plasmid from 1 ng/μl to 1 fg/μl. B) Ten-fold serial dilution of A. phagocytophilum total DNA from 1 pg/μl to 1 fg/μl. Lane N, non-template control (NTC, water); lane L, 100 bp DNA ladder.

FIG. 14. Sensitivity assays of A. marginale lateral flow RPA primers and probe. A) Ten-fold serial dilution of Artificial Positive Control (APC) from 1 ng/μl to 1 fg/μl. B) Ten-fold serial dilution of A. marginale total DNA from 0.01 ng/μl to 1 fg/μl. Lane C, flow-check line; Lane 2, detects FAM/Biotin labelled amplicons (A. marginale); Lane 1, detects DIG/Biotin labelled amplicons (GAPDH—internal control); Lane N, non-template control (NTC, water).

FIG. 15. Sensitivity assays of A. ovis lateral flow RPA primers and probe. A) Ten-fold serial dilution of Artificial Positive Control (APC) from 1 ng/μl to 1 fg/μl. B) Ten-fold serial dilution of A. marginale total DNA from 1 pg/μl to 1 fg/μl. Lane C, flow-check line; Lane 2, detects FAM/Biotin labelled amplicons (A. ovis); Lane 1, detects DIG/Biotin labelled amplicons (GAPDH—internal control); Lane N, non-template control (NTC, water).

FIG. 16. Sensitivity assays of A. phagocytophilum lateral flow RPA primers and probe. A) Ten-fold serial dilution of Artificial Positive Control (APC) from 1 ng/μl to 1 fg/μl. B) Ten-fold serial dilution of A. phagocytophilum total DNA from 1 ng/μl to 1 fg/μl. Lane C, flow-check line; Lane 2, detects FAM/Biotin labelled amplicons (A. ovis); Lane 1, detects DIG/Biotin labelled amplicons (GAPDH—internal control); Lane N, non-template control (NTC, water).

FIG. 17. A. marginale nfo RPA using positive serum samples. Lane C, flow-check line; Lane 2, detects FAM/Biotin labelled amplicons (A. marginale); Lane 1, detects DIG/Biotin labelled amplicons (GAPDH gene); 1-24, A. marginale positive serum samples; APC, Artificial positive control; Am, A. marginale positive reference control; NTC, non-template control (water).

FIG. 18. A. marginale nfo RPA using positive serum samples. Lane C, flow-check line; Lane 2, detects FAM/Biotin labelled amplicons (A. marginale); Lane 1, detects DIG/Biotin labelled amplicons (GAPDH gene); 1-25, A. marginale positive blood samples; APC, Artificial positive control; N, non-template control (water).

FIG. 19. A. phagocytophilum nfo RPA using positive cell culture samples. Lane C, flow-check line; Lane 2, detects FAM/Biotin labelled amplicons (A. phagocytophilum); Lane 1, detects DIG/Biotin labelled amplicons (GAPDH gene); 27-28, A. phagocytophilum positive cell culture samples; APC, Artificial positive control; NTC, non-template control (water).

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “polypeptide” as used herein will be understood to refer to a polymer of amino acids. The polymer may include d-, l-, or artificial variants of amino acids. In addition, the term “polypeptide” will be understood to include peptides, proteins, and glycoproteins.

The term “polynucleotide” as used herein will be understood to refer to a polymer of two or more nucleotides. Nucleotides, as used herein, will be understood to include deoxyribose nucleotides and/or ribose nucleotides, as well as artificial variants thereof. The term polynucleotide also includes single-stranded and double-stranded molecules.

The terms “analog” or “variant” as used herein will be understood to refer to a variation of the normal or standard form or the wild-type form of molecules. For polypeptides or polynucleotides, an analog may be a variant (polymorphism), a mutant, and/or a naturally or artificially chemically modified version of the wild-type polynucleotide (including combinations of the above). Such analogs may have higher, full, intermediate, or lower activity than the normal form of the molecule, or no activity at all. Alternatively and/or in addition thereto, for a chemical, an analog may be any structure that has the desired functionalities (including alterations or substitutions in the core moiety), even if comprised of different atoms or isomeric arrangements.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “pharmaceutically-acceptable excipient” refers to any carrier, vehicle, and/or diluent known in the art or otherwise contemplated herein that may improve solubility, deliverability, dispersion, stability, and/or conformational integrity of the compositions disclosed herein.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “child” is meant to refer to a human individual who would be recognized by one of skill in the art as an infant, toddler, etc., or an individual less than about 18 years of age, usually less than about 16 years of age, usually less than about 14 years of age, or even less (e.g., from newborn to about 2, about 3, about 4, about 5, about 6, about 7, about 8, about 9, about 10, about 11, or about 12 years of age). The term “elderly” generally refers to a human individual whose age is greater than about 50 years of age, usually greater than about 55 years of age, frequently greater than about 60 years of age or more (e.g., about 65 years of age and upwards).

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of pulmonary fibrosis. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the condition/disease/infection in conjunction with the pharmaceutical compositions of the present disclosure. This concurrent therapy can be sequential therapy, where the patient is treated first with one pharmaceutical composition and then the other pharmaceutical composition, or the two pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), certain non-limiting embodiments thereof are directed to kits, devices, systems, and methods for detecting species-specific Anaplasma infections in, for example (but not by way of limitation), livestock in the field and at the point of care. The kits, devices, systems, and methods rely on recombinase polymerase amplification (RPA) as an easy-to-use and rapid isothermal detection method. In addition, in certain non-limiting embodiments, the kits, devices, systems, and methods assays can identify and discriminate between three Anaplasma species.

Certain non-limiting embodiments of the present disclosure are directed to a kit that comprises three recombinase polymerase amplification (RPA) oligonucleotide pairs that each comprise a sense oligonucleotide (i.e., a primer and/or probe) and an antisense oligonucleotide (i.e., a primer and/or probe). The first recombinase polymerase amplification (RPA) oligonucleotide pair comprises a sense oligonucleotide (i.e., a primer and/or probe) and an antisense oligonucleotide (i.e., a primer and/or probe) for at least a portion of a major surface protein 4 (msp4) gene sequence from Anaplasma marginale; the second RPA oligonucleotide pair comprises a sense oligonucleotide (i.e., a primer and/or probe) and an antisense oligonucleotide (i.e., a primer and/or probe) for at least a portion of an msp4 gene sequence from Anaplasma ovis; and the third RPA oligonoucleotide pair comprising a sense oligonucleotide (i.e., a primer and/or probe) and an antisense oligonucleotide (i.e., a primer and/or probe) for at least a portion of an msp4 gene sequence from Anaplasma phagocytophilum. In addition, the first, second, and third RPA oligonucleotide pairs do not substantially cross react with the other species of Anaplasma.

The oligos (i.e., primers and/or probes) of the RPA oligonucleotide pairs may correspond to any portion of an Anaplasma msp4 gene sequence (or a variant or derivative thereof) and may have any structural characteristics or modifications known in the art, so long as the oligonucleotide pairs are able to function in accordance with the present disclosure.

For example (but not by way of limitation), each oligonucleotide of the first, second, and third RPA oligonucleotide pairs may be provided with any length that allows the oligonucleotides to amplify the corresponding template of the msp4 gene sequence and function in accordance with the present disclosure. Non-limiting examples of lengths that may be utilized in accordance with the present disclosure include about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45 nucleotides, and larger or smaller lengths. The scope of the present disclosure also explicitly includes ranges of lengths formed of two of any of the above values (i.e., a range of from about 30 nucleotides to about 35 nucleotides, etc.).

In addition, each oligonucleotide of the first, second, and third RPA oligonucleotide pairs may be provided with any G/C content that allows the oligonucleotide pair to amplify the corresponding template of the msp4 gene sequence and function in accordance with the present disclosure. Non-limiting examples of G/C contents that may be utilized in accordance with the present disclosure include about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, and about 70%, as well as higher or lower G/C contents. The scope of the present disclosure also explicitly includes ranges of G/C contents formed of two of any of the above values (i.e., a range of from about 40% to about 60%, etc.).

In addition, each oligonucleotide of the first, second, and third RPA oligonucleotide pairs may be provided with any Tm that allows the oligonucleotide pair to amplify the corresponding template of the msp4 gene sequence and function in accordance with the present disclosure. Non-limiting examples of Tm's that may be utilized in accordance with the present disclosure include about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., and about 110° C., as well as higher or lower Tm's. The scope of the present disclosure also explicitly includes ranges of Tm's formed of two of any of the above values (i.e., a range of from about 50° C. to about 100° C., etc.).

In certain non-limiting embodiments, the sense oligonucleotides may each contain a portion of a sequence selected from the msp4 sequences of one or more of SEQ ID NOS:25-64 (or a consensus sequence formed of two or more of SEQ ID NOS:25-64), while the antisense oligonucleotides may each have sequence that is complementary to a portion of a sequence selected from the msp4 sequences of one or more of SEQ ID NOS:25-64 (or a consensus sequence formed of two or more of SEQ ID NOS:25-64). That is, the sense oligonucleotide of the first RPA oligonucleotide pair may contain a portion of SEQ ID NO:26, while the antisense oligonucleotide of the first RPA oligonucleotide pair may have a sequence that is complementary to a portion of SEQ ID NO:26. Similarly, the sense oligonucleotide of the second RPA oligonucleotide pair may contain a portion of one or more of SEQ ID NOS:25 and 27-38 (or a consensus sequence formed of two or more of SEQ ID NOS:25 and 27-38), while the antisense oligonucleotide of the second RPA oligonucleotide pair may have a sequence that is complementary to a portion of one or more of SEQ ID NOS:25 and 27-38 (or a consensus sequence formed of two or more of SEQ ID NOS:25 and 27-38). Likewise, the sense oligonucleotide of the third RPA oligonucleotide pair may contain a portion of one or more of SEQ ID NOS:39-64 (or a consensus sequence formed of two or more of SEQ ID NOS:39-64), while the antisense oligonucleotide of the third RPA oligonucleotide pair may have a sequence that is complementary to a portion of one or more of SEQ ID NOS:39-64 (or a consensus sequence formed of two or more of SEQ ID NOS:39-64).

In certain particular (but non-limiting) embodiments, the first RPA oligonucleotide pairs have sequences represented by SEQ ID NOS: 1-2 or 9-10, the second RPA oligonucleotide pairs have sequences represented by SEQ ID NOS:3-4 or 11-12, and the third RPA oligonucleotide pairs have sequences represented by SEQ ID NOS:5-6 or 13-14.

In certain particular (but non-limiting) embodiments, at least one oligonucleotide from each RPA oligonucleotide pair may include a label for detection of the RPA product. A wide variety of nucleic acid labels useful in detecting an amplification product are well known in the art, and numerous nucleic acid labels are commercially available. Therefore, no further description thereof is deemed necessary.

Each of the oligonucleotides of an oligonucleotide pair may function as a primer and/or a probe, where the primer primes the synthesis of the reaction with the assistance of the pool of replicon-enzymes to generate millions of copies of the targeted region, while the probe labels each of these newly produced copies in a specific internal region to generate additive chemistry to each new copy generated, thereby allowing a sensitive detection proportional to the number of copies amplified. As such, when an oligonucleotide functions as a probe, the oligonucleotide may be modified, such as (but not limited to) by addition of a label, for detection of the RPA product. One or both of the oligonucleotides of each oligonucleotide pair may contain other modifications as necessary for improving the stability and/or yield of the assay and/or for improving the detection of the assay products. Such modifications to oligonucleotides for functioning as primers or probes are well known in the art, and thus no further discussion thereof is deemed necessary.

The RPA products generated by each of the first, second, and third RPA oligonucleotide pairs may be provided with any length that is acceptable under RPA parameters and that can be detected in accordance with the methods of the present disclosure. Non-limiting examples of product lengths that may be utilized in accordance with the present disclosure include about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, and about 250 base pairs, and larger or smaller base pair lengths. The scope of the present disclosure also explicitly includes ranges of lengths formed of two of any of the above values (i.e., a range of from about 100 base pairs to about 200 base pairs, etc.).

In certain non-limiting embodiments, the RPA product generated by the first RPA oligonucleotide pairs may comprise the sequence of SEQ ID NO:17 or 21. In certain non-limiting embodiments, the RPA product generated by the second RPA oligonucleotide pairs may comprise the sequence of SEQ ID NO:18 or 22. In certain non-limiting embodiments, the RPA product generated by the third RPA oligonucleotide pairs may comprise the sequence of SEQ ID NO:19 or 23.

The kits of the present disclosure may be provided with additional reagents that are used in the RPA reactions and/or detection assays. For example, but not by way of limitation, the kits may include one or more reagents such as (but not limited to), an RPA enzyme, betaine, magnesium acetate, rehydration buffer, nuclease-free water, and combinations thereof.

Also, the kits of the present disclosure may be provided with one or more RPA oligonucleotide pairs for conducting a fourth RPA reaction. In certain non-limiting embodiments, this additional RPA oligonucleotide pair may be for at least one positive or negative control. One non-limiting example of a positive control that can be utilized with the RPA primers and/or probes of the present disclosure includes RPA primers and/or probes for glyceraldehyde 3-phosphate dehydrogenase (GAPDH).

Further, the kits of the present disclosure may include other positive or negative controls, as known in the art. For example (but not by way of limitation), the kit may include an artificial positive control, as described in detail herein after.

In addition, the kits of the present disclosure may be provided with one or more components used in the RPA reactions and/or detection assays. For example, but not by way of limitation, the kits may include one or more of the following components: (i) a plurality of reaction chambers for performing a recombinase polymerase amplification reaction; (ii) a plurality of nucleic acid lateral flow assay devices; and/or (iii) at least one sample collection device.

Any reaction chambers known in the art as applicable for use with RPA may be utilized in accordance with the present disclosure. The reaction chambers may include one or more dried reagents for performing the RPA reactions. For example, the reaction chambers may comprise a dried reagent composition that includes a recombinase, a polymerase, and a single-stranded DNA binding protein. Non-limiting examples of reaction chambers that may be utilized in accordance with the present disclosure include those disclosed in U.S. Pat. Nos. 10,036,057 and 10,538,760.

Any nucleic acid lateral flow assay devices known in the art for detection of amplification products may be utilized in accordance with the present disclosure. Such devices are well known in the art and commercially available; two non-limiting examples thereof include Milenia HybriDetect lateral flow dipsticks (Milenia Biotec GmbH, GieRen, Germany) and PCRD nucleic acid lateral flow immunoassay (NALFIA) cassettes (Abingdon Health, York, UK). Non-limiting examples of nucleic acid lateral flow assay devices utilized in combination with RPA reaction chambers are disclosed in U.S. Pat. Nos. 10,036,057 and 10,538,760.

Any sample collection devices known in the art that are amenable for collecting samples for subsequent nucleic acid amplification assays can be utilized in accordance with the present disclosure. In one particular (but non-limiting) embodiment, the sample collection device is an elution independent collection device such as that disclosed in U.S. Pat. No. 9,423,398.

Certain non-limiting embodiments of the present disclosure are directed to systems that include various combinations of the kit components described herein above. For example (but not by way of limitation), certain non-limiting embodiments are directed to a system that includes any of the three recombinase polymerase amplification (RPA) oligonucleotide pairs for msp4 from Anaplasma marginale, Anaplasma ovis, and Anaplasma phagocytophilum as disclosed or otherwise contemplated herein in combination with a plurality of any of the reaction chambers disclosed or otherwise contemplated herein, a plurality of any of the nucleic acid lateral flow assay devices disclosed or otherwise contemplated herein, and/or at least one collection device. In a particular (but non-limiting) example, each reaction chamber comprises a reagent composition (such as, but not limited to, a dried reagent composition) comprising a recombinase, a polymerase, and a single-stranded DNA binding protein.

Certain non-limiting embodiments of the present disclosure are directed to a screening method the includes the steps of: obtaining a mammalian sample suspected of containing at least one species of Anaplasma; performing at least one RPA reaction with at least one of any of the RPA oligonucleotide pairs disclosed or otherwise contemplated herein; and determining if the species of Anaplasma for which the RPA oligonucleotide pair is specific is present in the sample based on the result of the at least one RPA reaction.

The method may further comprise the steps of: performing at least a second RPA reaction with at least another of any of the RPA oligonucleotide pairs disclosed or otherwise contemplated herein; and determining if the species of Anaplasma for which the RPA oligonucleotide pair is specific is present in the sample based on the result of the second RPA reaction.

The method may further comprise the steps of: performing at least a third RPA reaction with at least another of any of the RPA oligonucleotide pairs disclosed or otherwise contemplated herein; and determining if the species of Anaplasma for which the RPA oligonucleotide pair is specific is present in the sample based on the result of the third RPA reaction.

In certain non-limiting embodiments, the screening method discriminates between the presence of Anaplasma marginale, A. ovis, and A. phagocytophilum in the mammalian sample.

In certain non-limiting embodiments, each RPA reaction and detection assay involves the steps of: combining the mammalian sample with RPA primers and/or probes and an RPA reagent composition to provide a mixture and incubating the mixture under conditions that allow amplification to occur, wherein the RPA reagent composition comprises a recombinase, a polymerase, and a single-stranded DNA binding protein; contacting the incubated mixture with a nucleic acid lateral flow assay device; and detecting the RPA product via the nucleic acid lateral flow assay device.

Certain non-limiting embodiments of the present disclosure are directed to a method of selectively treating a subject. In the method, any of the screening methods disclosed or otherwise contemplated herein is performed, and it is determined that at least one species of Anaplasma is present in the sample based upon the detection of an Anaplasma species-specific msp4 gene in the sample; then, a treatment agent (such as, but not limited to, an antibiotic) is administered to the subject, wherein the treatment agent is effective against the at least one species of Anaplasma detected in the sample.

In certain non-limiting embodiments, all of the performing, determining, and administering steps occur in the field and/or at the point of care.

The method may be utilized to treat any subject that suffers from Anaplasma infections. Non-limiting examples of subjects that may benefit from such treatment include cows, sheep, and goats.

### Example

An Example is provided hereinbelow. However, the present disclosure is to be understood to not be limited in its application to the specific experimentation, results, and laboratory procedures disclosed herein. Rather, the Example is simply provided as one of various embodiments and is meant to be exemplary, not exhaustive.

The present Example is directed to the development and optimization of gel-based RPA and multiplex lateral flow RPA assays using DNA from experimental blood and serum samples infected with A. marginale and A. ovis, and culture cells containing A. phagocytophilum, which can be incorporated into a field-based testing protocol.

Materials and Methods

Source of bacteria and infected blood samples: Reference positive control consisting of frozen blood infected with Anaplasma marginale and A. ovis and cultured cells containing A. phagocytophilum in DMSO were provided by Oklahoma State University College of Veterinary Medicine, Stillwater (Dr. Kathy Kocan). Also, lyophilized DNA of A. ovis was provided by SaBio, Instituto de Investigación en Recursos Cinegéticos, Spain (Dr. Jose de la Fuente). Lab-reared ticks (Dermacentor variabilis) provided by the OSU Tick-Rearing Facility (Lisa Coburn) were used as negative control in the assays. A. marginale cELISA-positive serum samples from Oklahoma-based cattle were provided by Dr. Jerry Saliki (Oklahoma State University College of Veterinary Medicine, Stillwater).

RPA primer and probe design: The development of RPA primers for Anaplasma marginale, A. ovis, and A. phagocytophilum targeted the major surface protein 4 genes (msp4) as diagnostic target, and for internal control, the glyceraldehyde 3-phosphate dehydrogenase gene sequences occurred separately. Sequences were retrieved from the NCBI GenBank database and aligned using CLUSTALX2. The consensus sequences generated after the alignment were used to design the four primer pairs. RPA primers were designed using the web interface application Primer3 (Rozen and Skaletsky, 2000), the thermodynamics and tendency to form self-dimers by mFold (Arif and Ochoa-Corona, 2013). The RPA probes were designed by visual examination. The selected parameters for optimal RPA primers and probes as were described in the TwistAmp Design Manual (TwistDx, 2009) (Table 1). The in-silico specificity assay of primer sets were performed using BLASTn (Altschul et al., 1990). RPA primers and probes were synthesized by Integrated DNA Technologies (IDT) and Biosearch Technologies Inc., respectively.

TABLE 1
Parameters Selected for Optimal Performance of RPA Primers

| PARAMETERS | MINIMUM | | MAXIMUM | |
|---|---|---|---|---|
| Primer Length | 30 | nucleotides | 35 | nucleotides |
| GC Content | 40% | | 60% | |
| RPA Product Length | 100 | bp | 200 | bp |
| Primer Tm | 50° | C. | 100° | C. |

Artificial Positive Control (APC): Two artificial positive controls were designed, one based on tandem of forward and reverse complement sequences of RPA primers, and the second based on tandem of forward and reverse complement sequences of RPA primers and nfo probes targeting A. marginale, A. ovis, A. phagocytophilum and GAPDH gene as reported by Cassi et al. (2013). Each sequence was designed and made synthetically, then inserted into a restriction site of pUC57 (GenScript Inc, USA). According to Caasi et al. (2013), APC is a cloneable, synthetic, multi-target, and non-infectious control used for routine application in detection and diagnostics assays.

DNA isolation from blood, cell culture, and animal tissue: Dimethyl sulfoxide (DMSO) was removed from the A. marginale and A. ovis experimental blood isolates by incubating the samples at 55° C. for 5 min and centrifuging for 15 min at 8000 rpm. The supernatant was discarded, 200 μl of 1×PBS was added to the pellet, mixed thoroughly, and vortexed according to instructions from Oklahoma State University College of Veterinary Medicine, Stillwater (Dr. Kocan, personal communication). Total DNA was extracted from blood samples, cell cultures infected with A. phagocytophilum, and lab-reared ticks (Dermacentor variabilis) using the QIAmp Blood Mini kit (Qiagen, USA) and following the manufacturer's instructions.

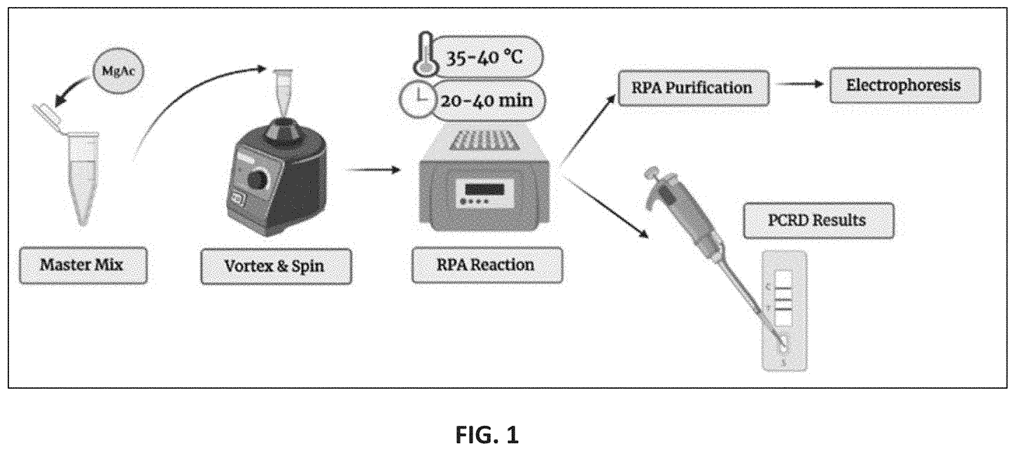

Optimization of basic and nfo RPA conditions: The RPA reaction was performed using the TwistAmp basic kit (TwistDx, UK). The reagents for the reaction were: 29.5 μl of rehydration buffer, 2.4 μl of each RPA forward and reverse primer (10 μM), with or without 10 μl betaine (5M; Thermo Fisher, USA), 3 μl DNA sample, nuclease-free water, and 2.5 μl of magnesium acetate (280 mM) to activate the reaction. The final volume of the reaction was 50 μl RPA reaction was performed in a dry bath incubator (GeneMate, USA). Incubation was at a constant temperature 35° C. to 40° C. for 20 min or 40 min, followed by 80° C. for 5 min to deactivate the enzyme complex. The amplified RPA product was purified by QIAquick PCR Purification Kit (Qiagen, USA) and analyzed by electrophoresis on a 2% agarose gel in 0.5×TAE buffer and SYBR safe (Invitrogen, USA) (FIG. 1).

The multiplex lateral flow RPA assay was performed using TwistAmp nfo kit (TwistDx, UK) as follows. The reagents for the reaction were: 29.5 μl of rehydration buffer, 10 μl betaine (5M), 0.6 μl of the probes (10 μM), 2.1 μl of each RPA forward and biotin-labeled reverse primers (10 μM), 3 μl DNA sample, and nuclease-free water. The final volume of the reaction was 50 μl Factorial combination between primers and probes of Anaplasma species and internal control (GAPDH) was developed in order to obtain the optimal primers/probes volume. The RPA reaction was performed in a dry bath incubator at 37° C. for 20 min (GeneMate, USA). At the end of the reaction, the temperature was increased to 80° C. for 5 min to deactivate the enzyme complex. After amplification, 6 μl of RPA product was mixed with 84 μl of buffer (Abingdon Health, UK). 75 μl of the diluted sample was added to a PCRD Nucleic Acid Detector cassette (Abingdon Health, UK). After 15 min, the results were read (FIG. 1).

Specificity of basic and nfo RPA assay: The specificity of the three RPA primer pairs was tested using A. marginale, A. ovis and A. phagocytophilum DNA reference positive controls. The non-infected tick DNA (negative control) and NTC (no template control) were included to confirm the reliability of the assay. All results were observed by electrophoresis (1×TAE) on agarose gel and lateral flow device.

Cloning of diagnostic fragments of Anaplasma marginale, A. ovis, and A. phagocytophilum: Three RPA primer sets were adapted to work in end-point PCR seeking cloning their diagnostic fragments. The A. marginale, A. ovis, and A. phagocytophilum PCR assay amplified products were purified from excised bands in agarose gel using QIAquick Gel Extraction Kit (Qiagen, USA). Then, TOPO TA cloning kit (Invitrogen, USA) was used for cloning the three amplified segments of Anaplasma according to the manufacturer's instructions. PCR products were inserted into the pCR′4-TOPO plasmid. Transformed plasmids were incubated in Escherichia coli competent cells following the manufacturer's protocol.

The amplified PCR products carried into transformed bacterial colonies were sequenced to verify whether the Anaplasma segments corresponding to each species were present in the plasmids. The bacterial plasmids were purified using Plasmid Mini Kit (Qiagen, USA) following the manufacturer's instructions.

Quantitative LC green PCR and quantification of Anaplasma DNA (plasmids): A. marginale, A. ovis, and A. phagocytophilum DNA blood samples were quantified using LC green qPCR analyses. Ten-fold dilutions of previous plasmid DNA, ranging from 1 ng/μl to 1 fg/μl were performed and used to plot the standard curve for three Anaplasma species DNA quantification. Plasmid DNA was measured by Qubit 4 Fluorometer (Thermo Fisher, USA). The qPCR amplification was carried out in 20 μl final volume containing 10 μl of One Taq Hot Start DNA Polymerase (Biolabs, USA), 0.5 μl of each RPA sense and antisense primers (10 μM), 2 μl of LC green (BioChem, USA), 1 μl of plasmid or DNA sample, and 6 μl nuclease-free water. Each reaction was tested in triplicate. The PCR cycling parameters were initial start of 50° C. for 3 min, initial denaturation of 94° C. for 4 min, 40 cycles of denaturation at 95° C. for 20 s, annealing at 62° C. for 20 s, extension at 72° C. for 20 s, and final extension at 72° C. for 4 min. The assays were performed in a Rotor Gene 6000 series (Corbett Research, USA). The mean of each set of replicates was calculated.

Sensitivity of basic and nfo RPA assay with plasmids and infected samples: Sensitivity was assessed using three plasmids carrying one fragment of A. marginale, A. ovis, and A. phagocytophilum each. The limit of detection for the three primer sets was determined using a ten-fold serial dilution of plasmid DNA and quantified Anaplasma DNA from 1 ng/μl to 1 fg/μl. One microliter of each dilution was used as template for basic and nfo RPA.

Screening of serum and blood samples using end-point PCR and RPA assays: Twenty-four A. marginale positive serum samples, twenty-five blood samples infected with A. marginale, and three cell culture infected with A. phagocytophilum were screened using published endpoint PCR (Torina et al., 2012), quantitative PCR with optimized RPA primers, and RPA reactions. Total DNA was extracted from each serum and blood samples. Both molecular techniques were performed using 3 μl of DNA. Twenty microliters of amplified PCR product were used for electrophoresis, 6 μl of amplified RPA product was mixed with 84 μl of lateral flow buffer, and 75 μl of the diluted sample was added to a PCRD Nucleic Acid Detector cassette (Abingdon Health, UK).

Results

RPA primer and probe design: The major surface protein 4 genes (msp4) sequences of A. ovis strain MD2059 (Accession number: DQ674249.1; SEQ ID NO:25) and A. marginale Brazil isolate (Accession number: AY714546.1; SEQ ID NO:26) were aligned seeking to design one pair of RPA primers in a region where the two species showed a significant difference. A. marginale msp4 sequence had a deletion of three nucleotides not traceable in A. ovis msp4 gene, which was found at nucleotide position 120. The RPA primers forward and reverse were designed from nucleotide position 101 to 131 and 174 to 203 of the A. marginale msp4 sequence, respectively. A. ovis RPA primers and nfo probe were designed based on consensus sequence of twelve available msp4 gene sequences from NCBI (Accession numbers: FJ460455.1, FJ460454.1, FJ460453.1, FJ460452.1, FJ460451.1, FJ460450.1, FJ460449.1, FJ460448.1, FJ460447.1, FJ460446.1, FJ460445.1, FJ460444.1 (SEQ ID NOS:27-38, respectively)). Primers and probe were located to anneal on nucleotide position 213-396.

Due to high variability among A. phagocytophilum strains, it was not possible to obtain a consensus sequence; however, a phylogenetic tree was generated using all the available msp4 gene from NCBI to select the close related sequences. Twenty-six sequences were aligned, and RPA primer set and nfo probe were designed based on the obtained consensus sequence (Accession numbers: KC847317.1, KP861635.1, KP861634.1, AY706390.1, AY706389.1, AY706388.1, AY706387.1, AY702925.1, MF974855.1, MF974854.1, HQ661163.1, HQ661162.1, HQ661159.1, HQ661158.1, HQ661157.1, HQ661156.1, HQ661155.1, HQ661154.1, AY829456.1, AY829455.1, AY530198.1, AY530197.1, AY530196.1, AY530195.1, AY530194.1, JQ522935.1 (SEQ ID NOS:39-64, respectively)). An in-silico specificity of Anaplasma spp. RPA primer sets and nfo probes using BLASTn showed 100% identity and 100% query coverage with each Anaplasma accessions available in the GenBank nucleotide database (NCBI). There were no matches detected among Anaplasma species as well as other bacteria.

The internal control was designed in the glyceraldehyde 3-phosphate dehydrogenase gene (GAPDH) of three available Bos taurus sequences (Accession numbers: NM_001034034.2, BC102589.1, XM_027541122.1 (SEQ ID NOS:65-67, respectively)) were aligned to generate a consensus sequence, which was used to design one set of RPA primers and nfo RPA located at nucleotide position 86-253. The in-silico specificity of GAPDH (internal control) RPA primer set and nfo probe showed 100% identity and 100% query coverage with mammalian species such as Bos taurus (cattle), Ovis aries (sheep), Capra hircus (goat), Odocoileus virginianus (deer) using BLASTn. No matches were detected among Anaplasma species.

The nfo probes were designed between forward and reverse primers. In order to adapt RPA reaction for lateral flow strip detection, probes contained three modifications: 6-carboxyfluorescein or digoxigenin tag at 5′, tetrahydrofuran located around 30 bp of the 5′-end, and a polymerase blocking group (C3 spacer) at the 3′-end. A. marginale, A. ovis, and A. phagocytophilum probes were labeled at the 5′ position with fluorescent dye FAM, GAPDH probe was labeled at 5′ position with DIG (Digoxigenin), and reverse primers with biotin. The sequences of primers and probes are listed in Tables 2 and 3.

TABLE 2
Anaplasma marginale, A. ovis, A. phagocytophilum,
and GADPH Gene (Internal Control) Basic RPA Primers

| | | | | | | | SEQ |
|---|---|---|---|---|---|---|---|
| Target | | | | | Product | | ID |
| Seq | Primer | Length | Tm | GC% | size | Sequence | NO: |
| A. marginale | Am3L_ | 33 | 76.63 | 54.55 | 103 | ACGAAGTGG | 1 |
| | msp4 | | | | | CTTCTGAAG | |
| | | | | | | GGGGAGTAA | |
| | | | | | | TGGGAG | |
| | Am3R_ | 30 | 72.99 | 50 | | GACTCACGC | 2 |
| | msp4 | | | | | ATGTCGAAC | |
| | | | | | | GAGGTAACA | |
| | | | | | | GAA | |
| A. | ApL_ | 30 | 75.14 | 60 | 202 | TGCGGCCGC | 3 |
| phagocytophilum | msp4 | | | | | AGTATGTGC | |
| | | | | | | CTGCTCCCT | |
| | | | | | | TTT | |
| | ApR_ | 30 | 62.31 | 40 | | GTTATAACC | 4 |
| | msp4 | | | | | TTTTACGTA | |
| | | | | | | AGATCCTCC | |
| | | | | | | CCT | |
| A. ovis | Ao2L_ | 35 | 67.75 | 45.71 | 184 | AGAGACCTC | 5 |
| | msp4 | | | | | GTATGTTAG | |
| | | | | | | AGGCTATGA | |
| | | | | | | CAAGAGTG | |
| | Ao2R_ | 35 | 68.57 | 48.57 | | CCTTCTGTA | 6 |
| | msp4 | | | | | GCTTGCTTC | |
| | | | | | | TAGTTCCAC | |
| | | | | | | TCTAGCTC | |
| GAPDH | GAPDH2_L | 30 | 72.53 | 50 | 168 | CTGAGACAA | 7 |
| gene | | | | | | GATGGTGAA | |
| | GAPDH2_R | 27 | 72.82 | 51.85 | | GGTCGGAGT | 8 |
| | | | | | | GAATTGCCG | |
| | | | | | | TGGGTGGAA | |
| | | | | | | TCATACTGG | |
| | | | | | | AAC | |

TABLE 3
Anaplasma marginale, A. ovis, A. phagocytophilum,
and GADPH Gene (Internal Control) nfo RPA Reverse
Primers and Probes

| | | | | | SEQ |
|---|---|---|---|---|---|
| | | | Product | | ID |
| Target Seq | Probe | Length | size | Sequence | NO: |
| A. marginale | Am3_probe | 39 | 103 | [5′Label- | 9 |
| | | | | FAM]TAGC | |
| | | | | TTTTACGT | |
| | | | | GGGTGCGG | |
| | | | | CCT[THF] | |
| | | | | CAGCCCAG | |
| | | | | CATTTCC | |
| | | | | [3′block- | |
| | | | | C3spacer] | |
| | Am3R_nfo | 30 | | [5′Label- | 10 |
| | | | | Biotin]GA | |
| | | | | CTCACGCAT | |
| | | | | GTCGAACGA | |
| | | | | GGTAACAG | |
| | | | | AA | |
| A. | ApR_probe | 50 | 202 | [5'Label- | 11 |
| phagocyto- | | | | FAM]TGATG | |
| philum | | | | CGTCTGATG | |
| | | | | TTAGCGGTG | |
| | | | | TTATGAACG | |
| | | | | G[THF]AGC | |
| | | | | TTTTACGTA | |
| | | | | AGTGGT[3' | |
| | | | | block- | |
| | | | | C3spacer] | |
| | ApR_nfo | 30 | | [5'Label- | 12 |
| | | | | Biotin] | |
| | | | | GTTATAAC | |
| | | | | CTTTTACG | |
| | | | | TAAGATCC | |
| | | | | TCCCCT | |
| A. ovis | Ao2_probe | 52 | 184 | [5'Label- | 13 |
| | | | | FAM]AAAT | |
| | | | | CCGGCTAC | |
| | | | | ACTTTTGC | |
| | | | | TTTCTCTA | |
| | | | | AGAA[THF] | |
| | | | | TTACTCAC | |
| | | | | ATCTTTCG | |
| | | | | A[3'block- | |
| | | | | C3spacer] | |

One non-limiting example of a 103 bp product produced using the primers of SEQ ID NOS:1-2 has been assigned SEQ ID NO:17. One non-limiting example of a 202 bp product produced using the primers of SEQ ID NOS:3-4 has been assigned SEQ ID NO:18. One non-limiting example of a 184 bp product produced using the primers of SEQ ID NOS:5-6 has been assigned SEQ ID NO:19. One non-limiting example of a 168 bp product produced using the primers of SEQ ID NOS:7-8 has been assigned SEQ ID NO:20. One non-limiting example of a product produced using the primers of SEQ ID NOS:9-10 has been assigned SEQ ID NO:21. One non-limiting example of a product produced using the primers of SEQ ID NOS:11-12 has been assigned SEQ ID NO:22. One non-limiting example of a product produced using the primers of SEQ ID NOS:13-14 has been assigned SEQ ID NO:23. One non-limiting example of a product produced using the primers of SEQ ID NOS:15-16 has been assigned SEQ ID NO:24.

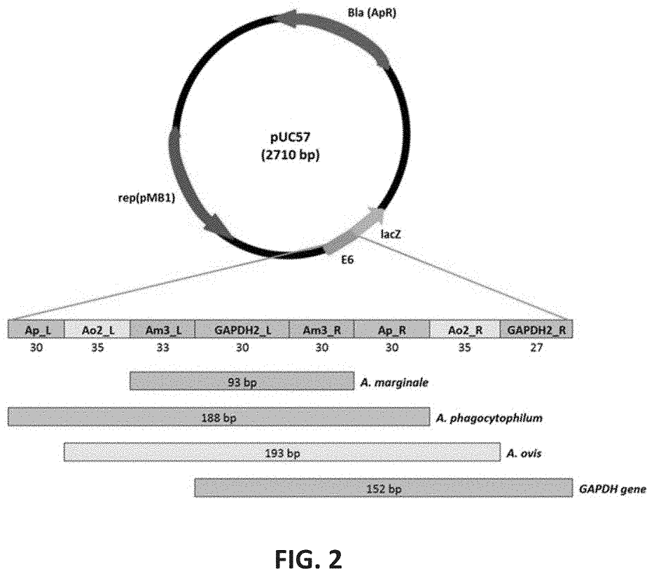

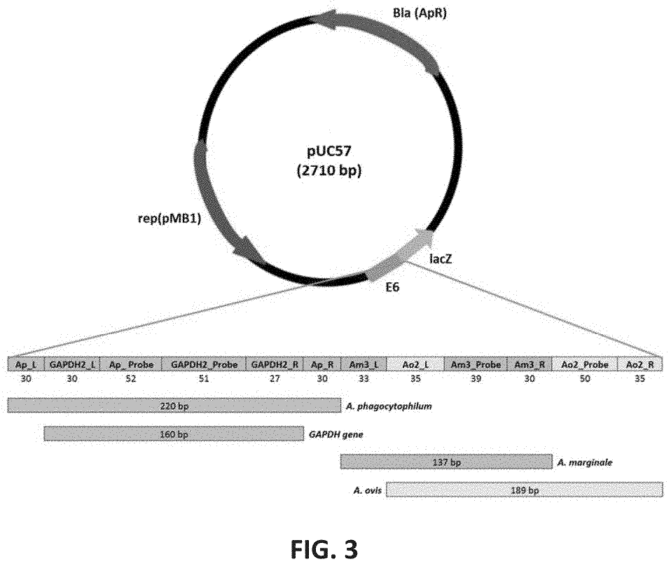

Artificial Positive Control: The two artificial positive controls were designed using the RPA forward primers, reverse complement primers, and nfo probe sequences inserted in pUC57 and cloned Escherichia coli competent cells (FIGS. 2 and 3). Endpoint PCR reactions targeting the whole APC sequences from bacterial colonies were made for each artificial positive control. Basic RPA APC showed the expected PCR product size of 250 bp using A. phagocytophilum forward (Ap_L) and GAPDH reverse (GAPDH2_R) primers. Lateral flow RPA APC showed the amplification of the expected PCR product size of 442 bp using A. phagocytophilum forward (Ap_L) primer and A. ovis reverse (Ao2_R) primer.

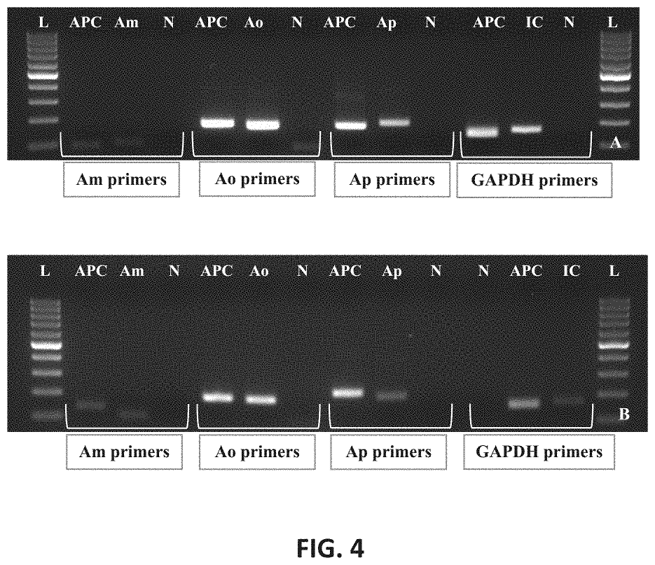

Furthermore, all targets of both APCs were amplified using basic RPA reactions in which the results were revealed by agarose gel electrophoresis. The product size between APCs and samples were slightly different. For instance, RPA amplicons generated using basic RPA_APC and A. marginale primers (Am3R/L), A. ovis primers (Ao2R/L), A. phagocytophilum primers (ApR/L), and GAPDH primers (GAPDH2R/L) were 93 bp, 193 bp, 188 bp, and 152 bp, respectively; while amplicons produced by A. marginale, A. ovis, A. phagocytophilum, and GAPDH samples were 103 bp, 184 bp, 202 bp, and 168 bp, respectively (FIG. 4, Panel A). In the case of nfo RPA APC, the expected RPA product sizes using this synthetic control were 137 bp, 189 bp, 220 bp, and 160 bp which were amplified using A. marginale primers (Am3R/L), A. ovis primers (Ao2R/L), A. phagocytophilum primers (ApR/L), and GAPDH primers (GAPDH2R/L), respectively (FIG. 4, Panel B).

In multiplex lateral flow RPA, primers and probes combinations were optimized in order to obtain same intensity of test lines.

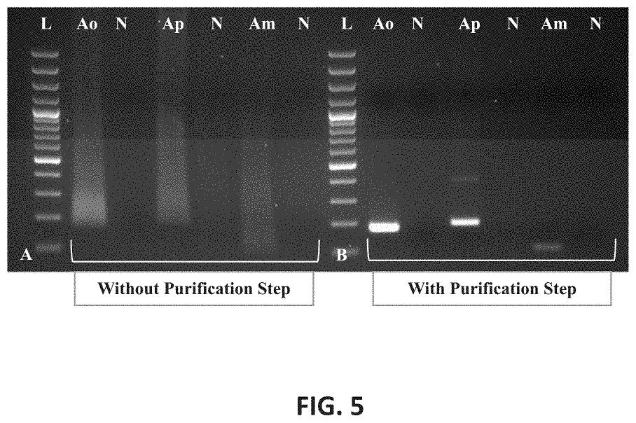

Optimization of basic and nfo RPA conditions: Extracted DNA from blood infected with Anaplasma marginale and A. ovis, and culture cells containing A. phagocytophilum were used as analyte for the optimization of basic RPA and nfo RPA reactions. The first assessed parameter evaluated was the purification step after the incubation at 39° C. for 40 min as recommended by manufacturer's protocol. The results revealed that the purification protocol allowed visualization of bands (FIG. 5, Panel B); otherwise, smears appeared when the purification step was not performed (FIG. 5, Panel A). Additionally, three reactions with bacterial DNA generated amplicons of expected size (A. ovis=184 bp, A. phagocytophilum=202 bp, and A. marginale=103 bp), whereas none of the blank controls was amplified.

Betaine (10 μl) was added to the RPA reaction to minimize false-positives and to reduce mis-priming. Results visualized on agarose gel confirmed non-template controls were not amplified when betaine was added to the reaction. However, false amplification may occur in RPA assays without this reagent. Furthermore, betaine did not interfere during amplification, and amplification was observed in the presence of betaine.

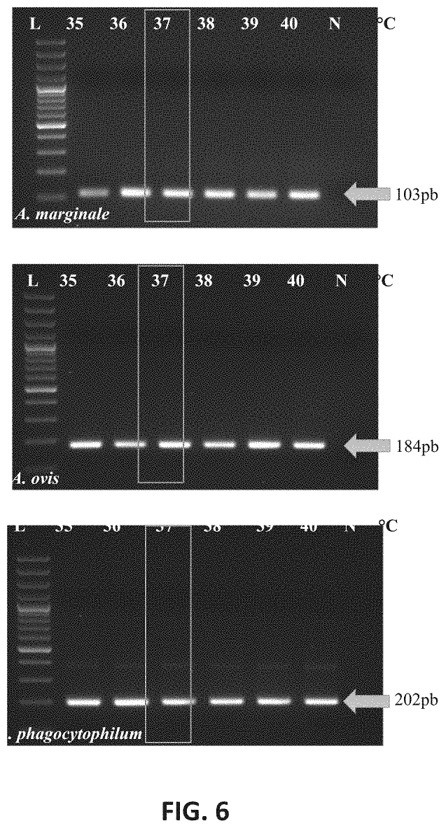

The three primer sets amplified the three expected diagnostic products from the predicted target of A. marginale (103 bp), A. ovis (184 bp), and A. phagocytophilum (202 bp) msp4 gene within a range of six temperatures: 35, 36, 37, 38, 39, and 40° C. This result indicates that each primer set performs well in a broad range of temperatures, as band intensity was almost the same in each of the assays; therefore, the selected temperature was 37° C. Anaplasma primer sets did not amplify products from non-template controls (water) (FIG. 6).

The reaction time was determined at 37° C., and 20 and 40 min were tested. No difference in band intensity was detected between the two reaction times; therefore, the target amplification can be done for either of these two time periods. The most suitable time for this isothermal reaction was considered to be 20 min.

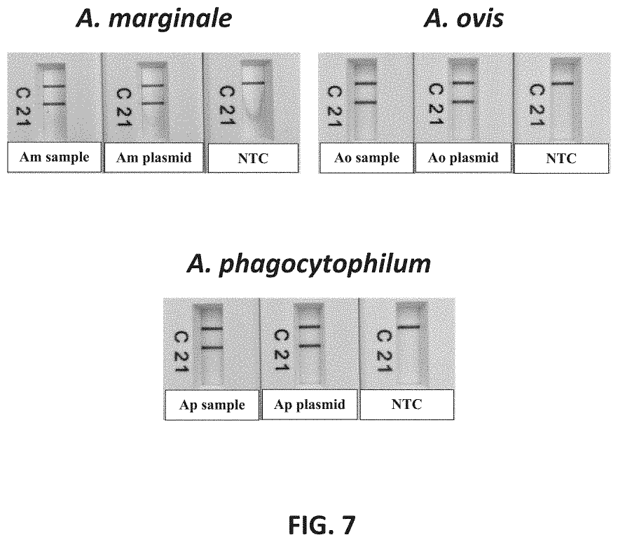

Betaine, temperature, and incubation time were optimized using basic RPA reactions from results observed in agarose gel. To monitor nfo RPA amplification (lateral flow), the assay was performed with the chemistry for TwistAmp nfo reaction, including forward primer, labeled probe, and labeled reverse primer. Lateral flow reaction in PCRD Nucleic Acid Detection strips were used to detect A. marginale, A. ovis, and A. phagocytophilum amplified products. The presence of control line C confirmed the lateral flow assay was working properly, as test line 2 consistently appeared in positive controls, sample, and plasmid. All tests were consistent of each assay, demonstrating the amplification of all bacterial targets tested, while only control line was observed in the negative control water (FIG. 7).

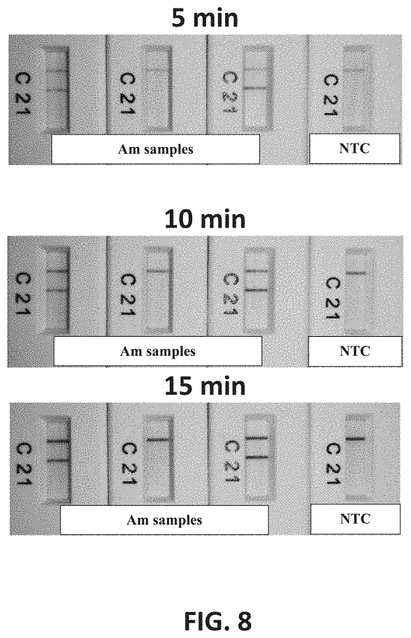

Faint test lines on the lateral flow dipstick were observed when the incubation time was 5 min. However, as the incubation time was increased to 10 min and 15 min, stronger positive signals at the test lines were visualized (FIG. 8). The nfo RPA reaction time included two-steps: first RPA amplification in dry bath incubator at 37° C. for 20 min, and second lateral flow dipstick at room temperature for 10 min as recommended.

Glyceraldehyde 3-phosphate dehydrogenase (GAPDH) gene is a conserved gene found in mammalian cells and has been used extensively as internal control for detection methods. The basic RPA reaction was performed using total DNA extracted from cattle blood infected with A. marginale and sheep blood infected with A. ovis. The RPA primer set GAPDH2R/L amplified a product from the predicted target of GAPDH gene at 37° C. for 20 min. The RPA products were obtained within the expected amplification size of 168 bp. The reverse and forward RPA primers did not amplify products from negative control (tick DNA) and non-template control (water).

Multiplex lateral flow RPA reactions were performed targeting each Anaplasma species with the GAPDH housekeeping gene (internal control). A factorial combination assay to include four volumes of primers (2.1 μl, 1.575 μl, 1.05 μl, and 0.525 μl) and probes (0.6 μl, 0.45 μl, 0.3 μl, and 0.15 μl) was evaluated in order to obtain the optimal combination between internal control and Anaplasma spp. Sixteen nfo RPA was developed using an Artificial Positive Control (APC: 1 ng/μl) as a template. The results demonstrated A. marginale and internal control test lines were clear and intense when primers were 1.8 μl (0.36 μM final concentration) and probes were 0.2 μl (0.04 μM final concentration). The best combination for A. ovis and A. phagocytophilum was 1.05 μl of primers (0.21 μM final concentration) and 0.6 μl of probes (0.12 μM final concentration), in which the two test bands (line 1 and 2) were equally intense and clear. No signal was observed in non-template control (water).

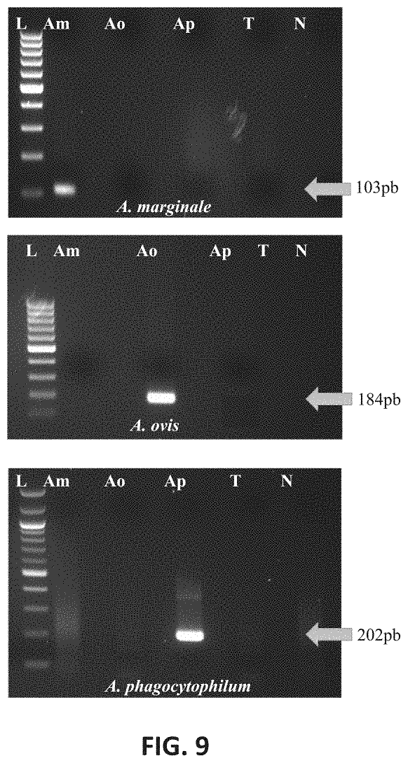

Specificity of basic and nfo RPA assay: Both RPA reactions (basic RPA-electrophoresis and nfo RPA-lateral flow dipsticks) were tested for specificity among the three Anaplasma species using genomic DNA of each bacteria. A. marginale, A. ovis, and A. phagocytophilum sense and antisense primers were tested. The single amplification of each primer set is shown, and no amplification was observed for the other two Anaplasma samples analyzed (FIG. 9). No cross-amplification was observed in each reaction. Lab-reared tick DNA as negative control and NTC (no template control) were included to confirm the reliability of each test. These negative controls did not produce any reaction using basic RPA reactions.

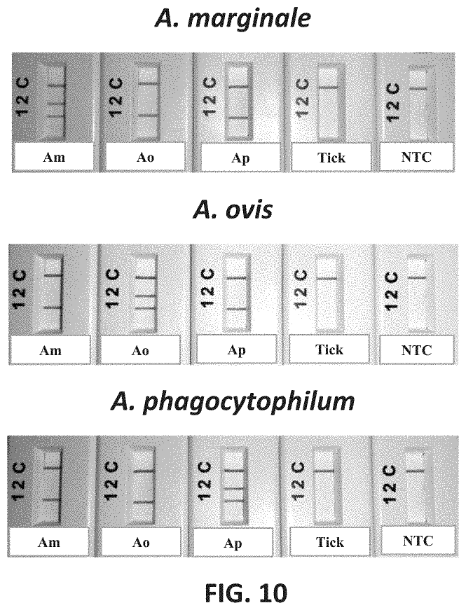

To evaluate the detection specificity of the established lateral flow RPA reaction, A. marginale, A. ovis, and A. phagocytophilum labeled reverse primers, forward primers, and nfo probes were tested; each assay included an internal control (GAPDH). Nfo RPA was positive only in each Anaplasma spp. target; a solid positive test band of internal control appeared in each Anaplasma DNA, whereas no signals (lines 1 and 2) were observed on the negative dipsticks (tick and NTC) (FIG. 10). The results indicated that the primer-probe combinations designed for lateral flow RPA reactions were specific to its corresponding targets. The isothermal reaction can detect and discriminate among Anaplasma species.

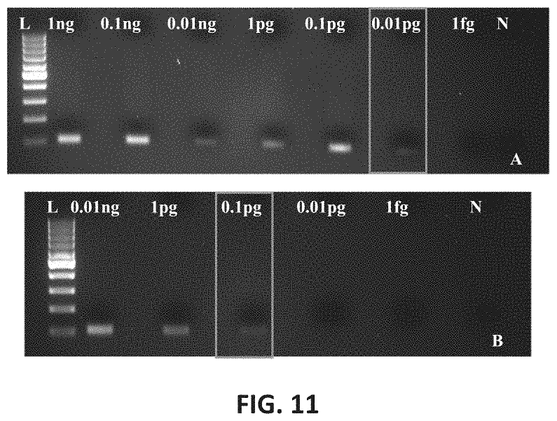

Sensitivity of basic and nfo RPA assay with plasmids and infected samples: The purified plasmid of A. marginale, A. ovis and A. phagocytophilum from 1 ng/μl to 1 fg/μl by serial dilution was used for threshold detection of the basic RPA method. The results demonstrated that basic RPA was able to detect until 0.01 pg/μl, 1 pg/μl, and 1 fg/μl using A. marginale, A. ovis, and A. phagocytophilum primers, respectively (Panel A of FIGS. 11, 12, and 13, respectively), when the amount of DNA was converted to copy number by the following formula (amount of DNA (ng)×6.022×10²³)/(length of DNA (bp)×10⁹×650) (Peng et al., 2019). The limit of detection in number of copies of A. marginale was 8.99×10⁴ copies, A. ovis was 5.04×10⁶ copies, and A. phagocytophilum was 4.59×10² copies.

A standard curve was used to determine the concentration of A. marginale, A. ovis, and A. phagocytophilum in total extracted blood DNA. The concentration of bacteria in the positive infected blood samples was A. marginale: 10.238 pg/μL, A. ovis: 1.252 pg/μL, and A. phagocytophilum: 13.084 ng/μl. Therefore, these three samples were used in basic and nfo RPA sensitivity assays. Additionally, three quantitative PCR showed a significant correlation (r=0.99).

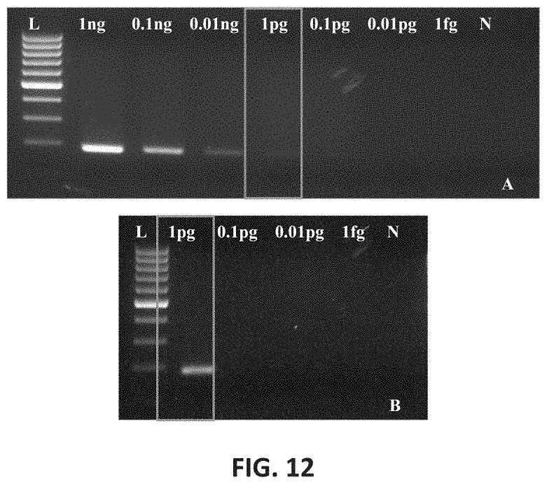

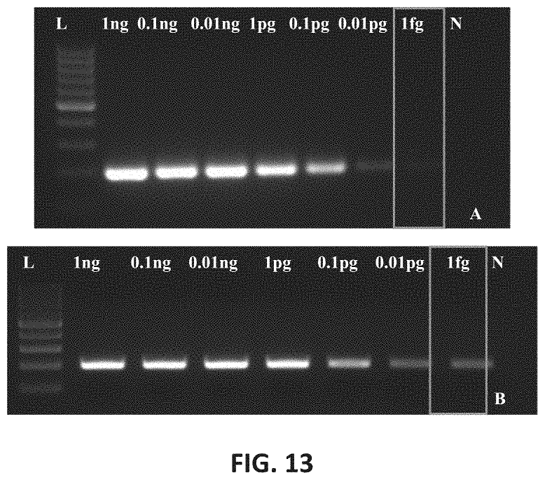

A. marginale quantified sample was diluted from 0.01 ng/μl to 1 fg/μ1, the limit of detection of basic RPA using total DNA was 0.1 pg/μl or 8.99×10⁵ copies (FIG. 11, Panel B). The analytical sensitivity of basic RPA to detect A. marginale from plasmid DNA was 10 times more sensitive than from an infected blood sample. This result could be due to inhibitors present in the blood sample or purification step after RPA incubation. A. ovis quantified sample was diluted from 1 pg/μl to 1 fg/μl, the limit of detection of basic RPA using total DNA was 1 pg/μl or 5.04×10⁶ copies (FIG. 12B). A. phagocytophilum quantified sample was diluted from 1 ng/μl to 1 fg/μ1, and the limit of detection of basic RPA using total DNA was 1 fg/μl or 4.59×10³ copies (FIG. 13, Panel B). The analytical sensitivity of basic RPA to detect A. phagocytophilum and A. ovis from plasmid DNA and an infected blood sample was equivalent. No amplification was observed with non-template control in each essay.

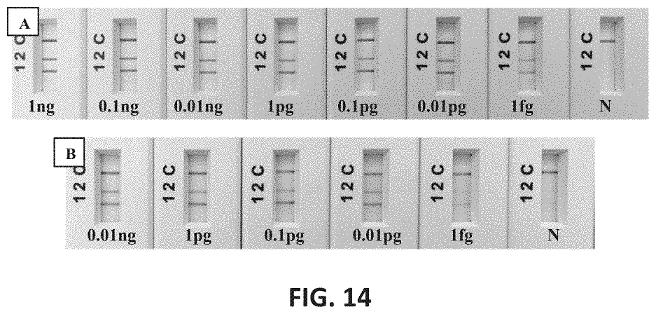

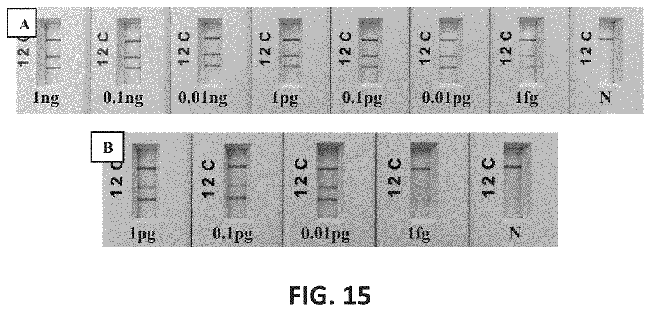

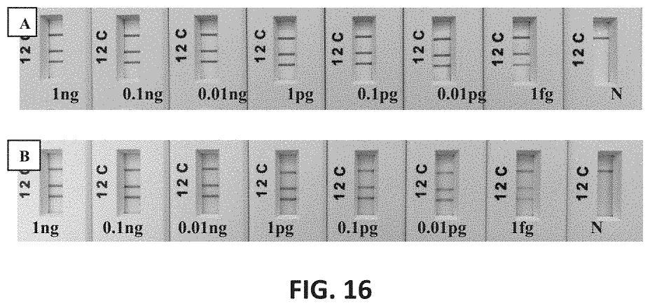

The detection threshold of lateral flow RPA assays was measured using a ten-fold serial dilution of the Artificial Positive Control (APC) from 1 ng/μl to 1 fg/μl. The results of A. marginale, A. ovis, and A. phagocytophilum RPA reactions using primers and probes demonstrate that the method allows for detection as low as 1 fg/μl with APC and bacterial measured total DNA (FIGS. 14, 15, and 16). In number of copies, the limit of detection was 8.99×10³ copies of A. marginale, 5.04×10³ copies of A. ovis, 4.59×10³ copies of A. phagocytophilum, and 5.51×10³ copies of internal control (GAPDH). Clear test (1, 2) and control lines appeared on each strip; however, 1 and 2 test lines were faint when DNA concentration was decreasing. Only the control line band was observed with non-template control (water) in each assay. Therefore, the analytical sensitivity of lateral flow RPA was higher than basic RPA amplification detected by agarose gel electrophoresis.

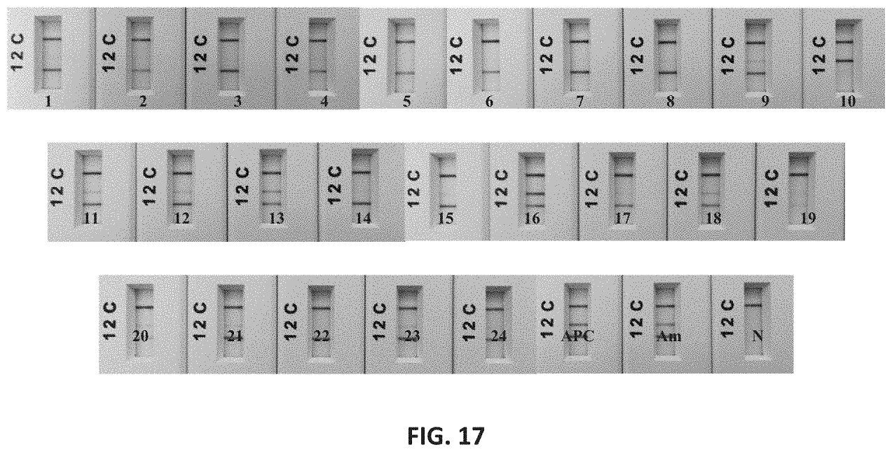

End-point PCR and RPA analyses using serum and blood samples: Twenty-four A. marginale positive serum samples were simultaneously detected by endpoint PCR previously described by Torina et al. (2012) and multiplex lateral flow RPA. In 24 samples, one sample tested positive by endpoint PCR, and 22 samples tested positive by A. marginale lateral flow RPA (Table 4 and FIG. 17). The difference between these two techniques is because the limit of detection of published PCR is 0.5 pg/μ1, while multiplex lateral flow RPA is 1 fg/μl. Therefore, in this Example, lateral flow RPA is more sensitive than published end point PCR. The internal control test line (1) appeared in each serum sample. A. marginale test lines (2) were faint and not clear in five serum samples (FIG. 17). No amplification was observed with non-template control (water) of both assays. Expected PCR product size of 344 bp was visible with A. marginale reference positive control. Two test lines (1, 2) were observed with APC and A. marginale reference positive control in PCRD cassette (FIG. 17). A. marginale lateral flow RPA assay was more sensitive than endpoint PCR.

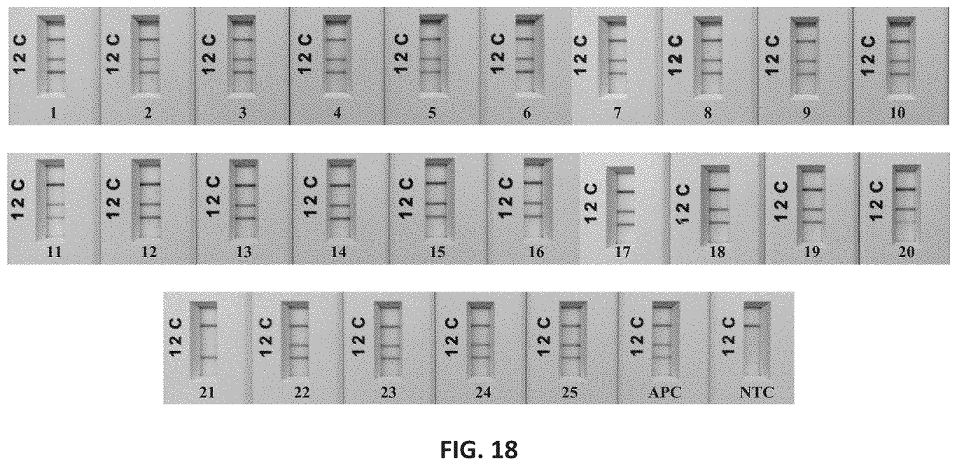

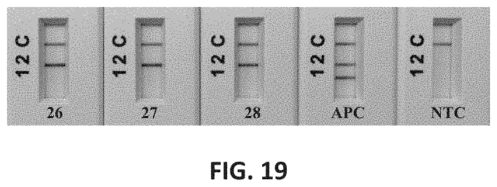

Twenty-five A. marginale positive blood and three A. phagocytophilum positive cell culture samples were simultaneously detected by qPCR using optimized RPA primers and multiplex lateral flow RPA (FIGS. 18-19). In 25 samples, 24 samples were positive for A. marginale by both diagnostic techniques. Table 5 shows the amount of quantified Anaplasma DNA in total extracted blood DNA. All cell culture samples tested positive for A. phagocytophilum by qPCR and nfo RPA reactions. No amplification was observed with non-template control (water) of both assays. Two test lines (1, 2) were observed with APC, A. marginale, and A. phagocytophilum reference positive control in PCRD cassette.

TABLE 4
Endpoint PCR and Multiplex Lateral Flow RPA Detection of
A. marginale from Serum Samples Provided by Oklahoma State
University College Of Veterinary Medicine (Stillwater, OK)

| | | Multiplex lateral flow RPA | |
|---|---|---|---|
| | A. marginale | A. marginale | Internal control |
| Serum Samples | PCR (published) | (Line 2) | (Line 1) |
| 1 | − | + | + |
| 2 | − | + | + |
| 3 | − | − | + |
| 4 | − | + | + |
| 5 | − | − | + |
| 6 | − | + | + |
| 7 | − | + | + |
| 8 | − | + | + |
| 9 | − | + | + |
| 10 | − | + | + |
| 11 | − | + | + |
| 12 | − | + | + |
| 13 | − | + | + |
| 14 | − | + | + |
| 15 | − | + | + |
| 16 | + | + | + |
| 17 | − | + | + |
| 18 | − | + | + |
| 19 | − | + | + |
| 20 | − | + | + |
| 21 | − | + | + |
| 22 | − | + | + |
| 23 | − | + | + |
| 24 | − | + | + |
| APC | NT | + | + |
| Am sample | + | + | + |
| NTC | − | − | − |

TABLE 5
Quantitative PCR Using Optimized RPA Primers and Multiplex
Lateral Flow RPA Detection of A. marginale and A. phagocytophilum
from Blood and Cell Culture Samples Provided by Oklahoma State
University College Of Veterinary Medicine

| | | | RPA Results | | qPCR (RPA |
|---|---|---|---|---|---|
| | | | Line 1 | Line 2 | primers) |
| N° | Code | Prevalence | (IC) | (Anaplasma) | (pg/μl) |

A. marginale

| | | | | | |
|---|---|---|---|---|---|
| 1 | PA407 | 16 | + | + | 5.36 |
| 2 | PA407 | 0.4 | + | + | 0.13 |
| 3 | PA407 | 2 | + | + | 0.59 |
| 4 | PA407 | 0 | + | + | 15.73 |
| 5 | PA412 | 21 | + | + | 12.44 |
| 6 | PA412 | 2 | + | + | 2.26 |
| 7 | PA415 | 57 | + | + | 13.6 |
| 8 | PA417 | 32 | + | + | 16.42 |
| 9 | PA417 | 2 | + | + | 0.82 |
| 10 | PA417 | 1 | + | + | 0.0863 |
| 11 | PA417 | 0.5 | + | + | 0.0643 |
| 12 | PA417 | 9 | + | + | 0.357 |
| 13 | PA418 | 43 | + | + | 0.546 |
| 14 | PA418 | 3 | + | + | 0.128 |
| 15 | PA418 | 2 | + | + | 0.0837 |

Other A. marginale strains

| | | | | | |
|---|---|---|---|---|---|
| 16 | PA414 | 44 | + | + | 5.533 |
| 17 | PA349 | 50 | + | + | 4.43 |
| 18 | PA413 | 8 | + | + | 5.54 |
| 19 | PA420 | 12 | + | + | 9.47 |
| 20 | PA428 | 9 | + | + | 0.231 |
| 21 | PA430 | 7 | + | − | 0 |
| 22 | PA443 | 20 | + | + | 1.208 |
| 23 | PA481 | 35 | + | + | 13.97 |
| 24 | PA482 | 9 | + | + | 11.942 |
| 25 | PA499 | 8 | + | + | 3.748 |

A. phagocytophilum

| | | | | | |
|---|---|---|---|---|---|
| 26 | HL-60 | cell culture | − | + | 79.35 |
| 27 | Ap74 | cell culture | − | + | 28.91 |
| 28 | ISE6 | cell culture | − | + | 24.45 |

### DISCUSSION

This Example describes the development of three RPA primer and probe sets for rapid, sensitive, reliable, and species-specific detection of A. marginale, A. ovis, and A. phagocytophilum by gel-based RPA and multiplex lateral flow RPA using GAPDH gene as internal control. First, multiplex nfo RPA assay was developed for the detection of three Anaplasma species. The assay detected 91.67% (22/24) infections from positive-serum by cELISA-tested cattle and 96% (24/25) infections from positive blood samples, while PCR only detected 4.17% (1/24). The fact that the nfo RPA detected DNA from serum samples tested by cELISA, which is the industry gold standard, demonstrates the effectiveness and high sensitivity of the nfo RPA assay of the present disclosure over PCR detecting low titer infections of Anaplasma in cattle. RPA assays are relatively easy to develop and produce rapid results in comparison to endpoint or quantitative PCRs. Others have developed single-use RPA assays to diagnose tick-borne diseases such as Rocky Mountain spotted fever (Qi et al., 2018), theileriosis (Yin et al., 2017; Hassan et al., 2018b), Lyme borreliosis (Liu et al., 2016), piroplasmosis (Hassan et al., 2018a), equine piroplasmosis (Lei et al., 2020), and Crimean-Congo Hemorrhagic fever (Bonney et al., 2017). The application of RPA in the diagnosis of A. phagocytophilum has been previous reported (Zhao et al., 2019; Jiang et al., 2020); however, there are no reported studies for A. marginale and A. ovis detection.

The development of accurate RPA reactions requires the design of specific primers and probes, because this isothermal reaction requires primers (30-35 nucleotides) and probes (46-52 nucleotides) which are longer than conventional PCR primers. However, there is no optimal software available to develop RPA primers. In this Example, RPA primers were designed by the web interface application Primer3 (Rozen and Skaletsky, 2000), and RPA probes were created manually according to the selection parameters for the optimal RPA primers and probes described in TwistAmp Design Manual (TwistDx, UK) (Table 1). Three sets of RPA primers and probes were designed based on major surface protein 4 gene (msp4) sequences of A. marginale, A. ovis, and A. phagocytophilum, which allowed detection and discrimination among the three Anaplasma species. The internal control consisted of one set of RPA primers and probe targeting the glyceraldehyde 3-phosphate dehydrogenase gene (GAPDH), a conserved gene most commonly used as housekeeping gene in mammalian cells (Barber et al., 2005).

According to basic RPA reaction assays, the best RPA primers were A. ovis and A. phagocytophilum, which generated intense and clear bands compared with bands generated by RPA primers for A. marginale and GAPDH internal control. When the RPA product size was around 100-170 bp, the intensity of the bands (A. marginale and GAPDH) were noticeably impacted. However, longer RPA products around 180-200 bp produced better bands in the assays (A. ovis and A. phagocytophilum) (Table 2).

Molecular assays such as PCR, LAMP, and RPA are used for detection of pathogens of interest. In order to perform a nucleic-acid molecular technique, positive controls are required to ensure that the test is detecting the pathogen and to reduce false-negative results. Positive controls are difficult to obtain because not all proteins or pathogens are available, or some pathogens are exotic and not commercially available (Smith et al., 2006). Infected samples are also used as positive controls; however, shipping and handling of these samples is risky and require permits (Nechvatal et al., 2008). Artificial positive control is a novel concept of customized synthetic DNA inserts based on linear arrays of primer sequences designed from a variety of organisms or targets important in detection, diagnostics, or research areas (Caasi et al., 2013). The application of artificial positive controls (APCs) can reduce risks associated with in vivo positive controls and improve reliability of molecular detection techniques.

The performance of basic and lateral flow RPA of two APCs based on species-specific RPA primers and probe sequences synthesized in pUC57 were compared with Anaplasma infected blood samples used as reference positive controls that showed accurate amplification. The amplicons generated by the RPA assay were varied slightly in size from those generated by the Anaplasma references positive controls. This was most likely due to differing annealing sites of target sequences in vivo and the distribution of primers and probe sequences in APC; however, these variations should not be a problem, because Anaplasma species and internal control targets were amplified, which demonstrated that the APCs were working correctly. The APC design has been reported using PCR primer sequences to mimic multiple pathogens (Caasi et al., 2013). This is the first study to use RPA primers and nfo probes to construct two synthetic DNA positive controls targeting A. marginale, A. ovis, A. phagocytophilum, and GAPDH housekeeping gene.

RPA is a relatively new technology; while many are beginning to work with the technology and scientific information is published, this Example highlights some aspects not normally covered by the scientific literature. For example, when RPA results are shown in agarose gel, the proteins and enzymes used in the RPA reaction result in the inappropriate migration of amplicons during electrophoresis, which generates smears instead of bands (Babu et al., 2017). In this Example, basic RPA products were purified using a commercial PCR purification kit which successfully provided visualized bands. In contrast, the purification step is not required during the nfo RPA reaction, as the results are observed using lateral flow dipstick. However, a quick centrifugation step was added after lateral flow RPA incubation for 5 min to sediment protein and enzyme components and to improve reliability. Another aspect not reported in most RPA studies is the need to inactivate the reaction upon completion. At the end of the RPA incubation, temperature was increased (80° C. for 5 min) to inactivate enzymes and prevent possible cross-contamination among reactions. While commonly reported in LAMP assays (Tomita et al., 2008; Shi et al., 2012), this Example demonstrated the need to inactivate RPA assays in certain non-limiting embodiments. Also differing with LAMP assays, RPA needs considerably less incubation time. In this assay, there was no difference between 20 and 40 min of reaction incubation, with an optimal RPA reaction time of 20 min for A. marginale, A. ovis, and A. phagocytophilum detection. The RPA assays were faster than conventional PCR and LAMP in incubation time, which take up to 120 min and 60-90 min, respectively (Kawahara et al., 2006; Lee et al., 2012; Torina et al., 2012b; Wen et al., 2016; Giglioti et al., 2019).

One of the issues that needed to be overcome during the development of the RPA assay involved non-specific amplification that occurred in the non-template control (FIG. 6). This non-specific amplification was eliminated using betaine. Betaine is a reagent used in molecular techniques such as PCR, LAMP, and RPA to prevent secondary structures when target sequences, primers, and probes have high GC content (Jensen et al., 2010). RPA primers and probes are long, and the GC percentage may vary between 40% and 60%, which would favor formation of hairpins and create false-positive results. The addition of betaine allowed positive results at a wide range of temperatures (35° C. to 40° C.). The optimal selected temperature is 37° C. for basic and nfo RPA assays, which can be performed in a dry bath incubator, as well as other heating devices like a water bath (Crannell et al., 2014; Zhao et al., 2019).

As one of the first studies to develop a multiplex RPA assay, the present disclosure identified the need to optimize the multiplex RPA primers and probes to avoid cross-interaction between dyes and primer/probe secondary structure or hairpin formation, which cause lower signal intensity in the test lines or false-positive results, respectively (Kim and Lee, 2017; Poulton and Webster, 2018). In this Example, two targets, A. marginale msp4 and GAPDH genes, A. ovis msp4 and GAPDH genes, and A. phagocytophilum msp4 and GAPDH genes, were tested in single multiplex lateral flow RPA reactions with different primer and probe combinations. Consequently, sixteen amplifications were performed to obtain the best combination, aiming for two clear, intense lines.

Successful field-focused detection assays need to be as simple to run as possible. Anaplasma lateral flow RPA amplification is a very rapid detection technique providing accurate results in approximately 35 or 40 min (20 minutes reaction and 10 minutes detection) and 30 min of DNA extraction using EICD prototype. This Example focused on the use of lateral flow detection method because it is simple to use, requires untrained personnel, and the results are easy to interpret. There are at least two types of commercially available strips that are used in most lateral flow devices to detect nfo RPA amplified products: Milenia HybriDetect lateral flow dipsticks (Milenia Biotec GmbH, GieRen, Germany) and PCRD nucleic acid lateral flow immunoassay (NALFIA) cassettes (Abingdon Health, York, UK). PCRD cassettes were more sensitive than Milenia HybriDetect dipsticks, and the incubation time using PCRD to detect DNA target was around 1 min (Poulton and Webster, 2018). Moreover, test lines of PCRD were consistent and clear compared with those of the Milenia HybriDetect system, which were non-uniformed and had variable intensity. According to the manufacturer's protocol, PCRD cassettes have a detection limit of 0.001 μg/ml DNA. In this Example, test lines were visible after 5 min of incubation time using PCRD dipsticks; however, bands became more intense over time. Therefore, the optimal incubation time using PCRD cassettes for best nfo RPA results was determined to be 10 min.

When developing diagnostic assays, it is important to choose the most sensitive detection technology that can be used within the constraints of the system that is being worked in. In the case of this Example, it was highly important to have a detection assay that can detect the presence of Anaplasma DNA in a small quantity as possible, because many chronically-infected cattle have infections which are below the threshold of detection for some assays. Anaplasmosis can be detected by traditional and novel molecular methods which have reported different limits of detection. For example, endpoint PCR described by Torina et al., (2012b) can detect until 0.5 pg/μl of A. marginale and 0.005 pg/μl of A. ovis, while the limit of detection of a LAMP assay for A. marginale, A. ovis, and A. phagocytophilum is 1 fg/μl (Wen et al., 2016). In the current Example, the limit of detection of gel-based RPA assays for A. marginale, A. ovis, and A. phagocytophilum was 0.1 pg/μl, 1 pg/μ1, and 1 fg/μ1, respectively. The analytical sensitivity of multiplex lateral flow RPA was 1 fg/μl detecting three Anaplasma spp. and GADPH gene. High sensitivity was found using nfo RPA due to the use of FAM-labeled or DIG-labeled probes and Biotin-labeled reverse primer, which are joined to gold-labeled anti-FAM and anti-Biotin antibodies; therefore, more RPA product is captured (Hou et al., 2017). Also, biotin-labeled DNA can join with streptavidin linked by three or four molecules to generate more intense signal (Kim et al., 2011). In contrast, the limit of detection in gel-based RPA assays decreased because of the additional purification step after incubation. According to Dugan et al. (2002), 95% of DNA is recovered after PCR purification protocol using spin columns; hence, 5% of RPA products could be lost during this step. In summary, multiplex lateral flow RPA is more sensitive than conventional PCR. Both LAMP and RPA assays are highly sensitive, but RPA is faster and easier to observe the results than LAMP amplification.

### CONCLUSION

In conclusion, a molecular assay able to detect low levels of Anaplasma DNA in field-collected blood samples taken from cattle and sheep has been developed. It is also important to keep in mind that dual infections are common in different parts of the world, as co-infections of two or more Anaplasma species have been reported in ticks, deer, and cattle (De La Fuente et al., 2005; Aubry and Geale, 2011). In this Example, specificity assays of each set of RPA primers and probes were tested with each detecting one specific Anaplasma infected sample in each assay; i.e., A. marginale (Am3R/L), A. ovis (Ao2R/L), and A. phagocytophilum (ApR/L) primers only recognized their specific target and did not cross-react with the other two targets. The same results were observed in multiplex lateral flow RPA reactions. No cross-reactions were produced among Anaplasma species. Both gel-based RPA and lateral flow RPA identified and discriminated among three Anaplasma species and detected Anaplasma marginale DNA in the serum of 92% of cELISA-positive cattle and 96% of A. marginale-positive blood samples. By combining these species-specific RPA assays for three Anaplasma species with appropriate controls in a lateral-flow delivery system, the flexibility and utility of this molecular technique in the development of many types of field-diagnostics has been demonstrated.

While the attached disclosures describe the inventive concept(s) in conjunction with the specific drawings, experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

### REFERENCES

The following references, to the extent that they provide exemplary procedural or other details supplementary to those set forth herein, are specifically incorporated herein by reference. In addition, the following is not intended to be an Information Disclosure Statement; rather, an Information Disclosure Statement in accordance with the provisions of 37 CFR § 1.97 will be submitted separately.
Altschul, S. F., W. Gish, W. Miller, E. W. Myers, and D. J. Lipman. 1990. Basic local alignment search tool. Journal of Molecular Biology 215: 403-410.
Arif, M., and F. Ochoa-Corona. 2013. Comparative assessment of 5′ A/T-rich overhang sequences with optimal and sub-optimal primers to increase PCR yields and sensitivity. Molecular Biotechnology 55: 17-26.
Atif, F. A. 2015. Anaplasma marginale and Anaplasma phagocytophilum: Rickettsiales pathogens of veterinary and public health significance. Parasitology Research 114: 3941-3957.
Aubry, P., and D. Geale. 2011. A review of bovine anaplasmosis. Transboundary and Emerging Diseases 58: 1-30.
Babu, B., B. K. Washburn, S. H. Miller, K. Poduch, T. Sarigul, G. W. Knox, F. M. Ochoa-Corona, and M. L. Paret. 2017. A rapid assay for detection of Rose rosette virus using reverse transcription-recombinase polymerase amplification using multiple gene targets. J Journal of Virological Methods 240: 78-84.
Barber, R. D., D. W. Harmer, R. A. Coleman, and B. J. Clark. 2005. GAPDH as a housekeeping gene: analysis of GAPDH mRNA expression in a panel of 72 human tissues. J Physiological Genomics 21: 389-395.
Bonney, L. C., R. J. Watson, B. Afrough, M. Mullojonova, V. Dzhuraeva, F. Tishkova, and R. J. P. n. t. d. Hewson. 2017. A recombinase polymerase amplification assay for rapid detection of Crimean-Congo Haemorrhagic fever Virus infection. J PLoS Neglected Tropical Diseases 11: e0006013.
Caasi, D. R. J., M. Arif, M. Payton, U. Melcher, L. Winder, and F. M. Ochoa-Corona. 2013. A multi-target, non-infectious and clonable artificial positive control for routine PCR-based assays. J Journal of Microbiological Methods 95: 229-234.
Crannell, Z. A., B. Rohrman, and R. J. P. o. Richards-Kortum. 2014. Equipment-free incubation of recombinase polymerase amplification reactions using body heat. J PloS One 9.
Daher, R. K., G. Stewart, M. Boissinot, and M. G. Bergeron. 2016. Recombinase Polymerase Amplification for Diagnostic Applications. Clinical Chemistry 62: 947-958.
De La Fuente, J., V. Naranjo, F. Ruiz-Fons, U. Hofle, I. G. Fernandez De Mera, D. Villanúa, C. Almazán, A. Torina, S. Caracappa, and K. M. Kocan. 2005. Potential vertebrate reservoir hosts and invertebrate vectors of Anaplasma marginale and A. phagocytophilum in central Spain. J Vector-Borne Zoonotic Diseases 5: 390-401.
De Waal, D. T. 2000. Anaplasmosis control and diagnosis in South Africa. Annals of the New York Academy of Sciences 916: 474-483.
Dugan, K. A., H. S. Lawrence, D. R. Hares, C. L. Fisher, and B. J. J. o. F. S. Budowle. 2002. An improved method for post-PCR purification for mtDNA sequence analysis. J Journal of Forensic Science 47: 1-8.
Giglioti, R., C. C. Bassetto, C. H. Okino, H. N. de Oliveira, and M. C. de Sena Oliveira. 2019. Development of a loop-mediated isothermal amplification (LAMP) assay for the detection of Anaplasma marginale. Experimental and Applied Acarology 77: 65-72.
Hassan, M. A., J. Liu, M. S. Sajid, A. Mahmood, S. Zhao, Q. Abbas, G. Guan, H. Yin, and J. J. J. o. P. Luo. 2018a. Molecular detection of Theileria annulata in cattle from different regions of Punjab, Pakistan, by using recombinase polymerase amplification and polymerase chain reaction. J Journal of Parasitology 104: 196-201.
Hassan, M. A., J. Liu, M. S. Sajid, M. Rashid, A. Mahmood, Q. Abbas, G. Guan, H. Yin, J. J. T. Luo, and t.-b. diseases. 2018b. Simultaneous detection of Theileria annulata and Theileria orientalis infections using recombinase polymerase amplification. J Ticks Tick-borne Diseases 9: 1002-1005.
Hornok, S., A. Micsutka, I. F. De Mera, M. L. Meli, E. Gönczi, B. Tánczos, A. J. Mangold, R. Farkas, H. Lutz, and R. Hofmann-Lehmann. 2012. Fatal bovine anaplasmosis in a herd with new genotypes of Anaplasma marginale, Anaplasma ovis and concurrent haemoplasmosis. J Research in Veterinary Science 92: 30-35.
Hou, P., H. Wang, G. Zhao, C. He, and H. J. B. v. r. He. 2017. Rapid detection of infectious bovine Rhinotracheitis virus using recombinase polymerase amplification assays. J BMC Veterinary Research 13: 386.
James, A., and J. Macdonald. 2015. Recombinase polymerase amplification: emergence as a critical molecular technology for rapid, low-resource diagnostics. J Expert Review of Molecular Diagnostics 15: 1475-1489.
Jensen, M. A., M. Fukushima, and R. W. J. Davis. 2010. DMSO and betaine greatly improve amplification of GC-rich constructs in de novo synthesis. J PLoS One 5.
Jiang, L., P. Ching, C.-C. Chao, J. S. Dumler, and W.-M. Ching. 2020. Development of a sensitive and rapid recombinase polymerase amplification assay for detection of Anaplasma phagocytophilum. J Journal of Clinical Microbiology 58.
Kawahara, M., Y. Rikihisa, Q. Lin, E. Isogai, K. Tahara, A. Itagaki, Y. Hiramitsu, and T. J. A. E. M. Tajima. 2006. Novel genetic variants of Anaplasma phagocytophilum, Anaplasma bovis, Anaplasma centrale, and a novel Ehrlichia sp. in wild deer and ticks on two major islands in Japan. J Appl. Environ. Microbiol. 72: 1102-1109.
Kim, J. Y., and J.-L. Lee. 2017. Development of a multiplex real-time recombinase polymerase amplification (RPA) assay for rapid quantitative detection of Campylobacter coli and jejuni from eggs and chicken products. J Food Control 73: 1247-1255.
Kim, K. T., C. B. Chae 2011. Dramatic increase in the signal and sensitivity of detection via self-assembly of branched DNA. Molecules and Cells 3: 367-374.
Lee, C., Y. Lin, C. Tsang, and Y. Chung. 2012. A loop-mediated isothermal amplification (LAMP) assay for rapid detection of Anaplasma phagocytophilum infection in dogs. Turkish Journal of Veterinary and Animal Sciences 36: 205-210.
Lei, R., X. Wang, D. Zhang, Y. Liu, Q. Chen, and N. Jiang. 2020. Rapid isothermal duplex real-time recombinase polymerase amplification (RPA) assay for the diagnosis of equine piroplasmosis. J Scientific Reports 10: 1-11.
Lillis, L., J. Siverson, A. Lee, J. Cantera, M. Parker, O. Piepenburg, D. A. Lehman, and D. S. Boyle. 2016. Factors influencing recombinase polymerase amplification (RPA) assay outcomes at point of care. J Molecular Cellular Probes 30: 74-78.
Liu, W., H.-X. Liu, L. Zhang, X.-X. Hou, K.-L. Wan, and Q. Hao. 2016. A novel isothermal assay of Borrelia burgdorferi by recombinase polymerase amplification with lateral flow detection. International Journal of Molecular Sciences 17: 1250.
Ma, M., Z. Liu, M. Sun, J. Yang, G. Guan, Y. Li, J. Luo, and H. Yin. 2011. Development and evaluation of a loop-mediated isothermal amplification method for rapid detection of Anaplasma ovis. Journal of Clinical Microbiology 49: 2143-2146.
Nechvatal, J. M., J. L. Ram, M. D. Basson, P. Namprachan, S. R. Niec, K. Z. Badsha, L. H. Matherly, A. P. Majumdar, and I. Kato. 2008. Fecal collection, ambient preservation, and DNA extraction for PCR amplification of bacterial and human markers from human feces. Journal of Microbiological Methods 72: 124-132.
Notomi, T., H. Okayama, H. Masubuchi, T. Yonekawa, K. Watanabe, N. Amino, and T. Hase. 2000. Loop-mediated isothermal amplification of DNA. Nucleic Acids Research 28: e63-e63.
Pai, N. P., C. Vadnais, C. Denkinger, N. Engel, and M. Pai. 2012. Point-of-care testing for infectious diseases: diversity, complexity, and barriers in low- and middle-income countries. J PLoS Medicine 9.
Pan, L., L. Zhang, G. Wang, Q. Liu, Y. Yu, S. Wang, H. Yu, and J. He. 2011. Rapid, simple, and sensitive detection of Anaplasma phagocytophilum by loop-mediated isothermal amplification of the msp2 gene. Journal of Clinical Microbiology 49: 4117-4120.
Peng, Y., X. Zheng, B. Kan, W. Li, W. Zhang, T. Jiang, J. Lu, and A. Qin. 2019. Rapid detection of Burkholderia pseudomallei with a lateral flow recombinase polymerase amplification assay. J PloS One 14.
Poulton, K., and B. Webster. 2018. Development of a lateral flow recombinase polymerase assay for the diagnosis of Schistosoma mansoni infections. J Analytical Biochemistry 546: 65-71.
Qi, Y., Y. Shao, J. Rao, W. Shen, Q. Yin, X. Li, H. Chen, J. Li, W. Zeng, and S. Zheng. 2018. Development of a rapid and visual detection method for Rickettsia rickettsii combining recombinase polymerase assay with lateral flow test. J PloS One 13.
Rozen, S., and H. Skaletsky. 2000. Primer3 on the WWW for general users and for biologist programmers, pp. 365-386, Bioinformatics Methods and Protocols. Springer.
Shi, Y., H. Dong, R. Wang, L. Zhang, C. Ning, and F. Jian. 2012. Exploration of Loop-mediated Isothermal Amplification (LAMP) for detection of Cryptosporidium parvum. Chinese Journal of Zoonoses 12.
Smith, G., I. Smith, B. Harrower, D. Warrilow, and C. Bletchly. 2006. A simple method for preparing synthetic controls for conventional and real-time PCR for the identification of endemic and exotic disease agents. Journal of Virological Methods 135: 229-234.
Tomita, N., Y. Mori, H. Kanda, and T. Notomi. 2008. Loop-mediated isothermal amplification (LAMP) of gene sequences and simple visual detection of products. J Nature Protocols 3: 877-882.
Torina, A., A. Agnone, V. Blanda, A. Alongi, R. D'Agostino, S. Caracappa, A. M. Marino, V. Di Marco, and J. de la Fuente. 2012. Development and validation of two PCR tests for the detection of and differentiation between Anaplasma ovis and Anaplasma marginale. Ticks and tick-borne diseases 3: 283-287.
TwistDx. 2009. Appendix to the TwistAmp TM reaction kit manuals. TwistDx Cambridge, UK.
Wen, X., H. Jiang, Y. Zhang, X. Lang, J. Liu, and H. Ni. 2016. Rapid and sensitive diagnosis of cattle anaplasmosis by loop-mediated isothermal amplification (LAMP). Pak Vet J 36: 174-178.
Yin, F., J. Liu, A. Liu, Y. Li, J. Luo, G. Guan, and H. Yin. 2017. Rapid diagnosis of Theileria annulata by recombinase polymerase amplification combined with a lateral flow strip (LF-RPA) in epidemic regions. J Veterinary Parasitology 237: 125-129.
Zhao, S., Y. Cui, J. Jing, Y. Yan, Y. Peng, K. Shi, K. Wang, Y. Zhou, F. Jian, and L. Zhang. 2019. Rapid and sensitive detection of Anaplasma phagocytophilum using a newly developed recombinase polymerase amplification assay. J Experimental Parasitology 201: 21-25.

What is claimed is:

1. A kit, comprising:
a first recombinase polymerase amplification (RPA) oligonucleotide pair comprising a sense oligonucleotide and an antisense oligonucleotide for at least a portion of a major surface protein 4 (msp4) gene sequence from Anaplasma marginale;
a second RPA oligonucleotide pair comprising a sense oligonucleotide and an antisense oligonucleotide for at least a portion of an msp4 gene sequence from Anaplasma ovis; and
a third RPA oligonucleotide pair comprising a sense oligonucleotide and an antisense oligonucleotide for at least a portion of an msp4 gene sequence from Anaplasma phagocytophilum; and
wherein the first, second, and third RPA oligonucleotide pairs do not substantially cross react with the other species of Anaplasma.

2. The kit of claim 1, wherein each oligonucleotide of the first, second, and third RPA oligonucleotide pairs has a length in a range of from about 30 nucleotides to about 35 nucleotides, a G/C content in a range of from about 40% to about 60%, and a Tm in a range of from about 50° C. to about 100° C.

3. The kit of claim 1, wherein the first RPA oligonucleotide pairs have sequences represented by SEQ ID NOS: 1-2 or 9-10, the second RPA oligonucleotide pairs have sequences represented by SEQ ID NOS:3-4 or 11-12, and the third RPA oligonucleotide pairs have sequences represented by SEQ ID NOS:5-6 or 13-14.

4. The kit of claim 1, wherein at least one oligonucleotide from each RPA oligonucleotide pair is labeled.

5. The kit of claim 1, wherein an RPA product generated by each of the first, second, and third RPA oligonucleotide pairs has a length in a range of from about 100 base pairs to about 200 base pairs.

6. The kit of claim 1, further comprising at least one reagent selected from the group consisting of an RPA enzyme, betaine, magnesium acetate, rehydration buffer, nuclease-free water, and combinations thereof.

7. The kit of claim 1, further comprising a plurality of reaction chambers, each for performing a recombinase polymerase amplification reaction.

8. The kit of claim 7, wherein each reaction chamber comprises a dried reagent composition, wherein the dried reagent composition comprises a recombinase, a polymerase, and a single-stranded DNA binding protein.

9. The kit of claim 1, further comprising an RPA oligonucleotide pair for at least one positive control.

10. The kit of claim 9, wherein the positive control is glyceraldehyde 3-phosphate dehydrogenase (GAPDH).

11. The kit of claim 1, further comprising an artificial positive control.

12. The kit of claim 1, further comprising a plurality of nucleic acid lateral flow assay devices.

13. The kit of claim 1, further comprising at least one collection device.

14. The kit of claim 13, wherein the at least one collection device is an elution independent collection device.

15. A system, comprising:
three recombinase polymerase amplification (RPA) oligonucleotide pairs, each comprising a sense oligonucleotide and an antisense oligonucleotide for at least a portion of a major surface protein 4 (msp4) gene sequence, wherein the first RPA oligonucleotide pair is for msp4 from Anaplasma marginale, the second RPA oligonucleotide pair is for msp4 from Anaplasma ovis, and the third RPA oligonucleotide pair is for msp4 from Anaplasma phagocytophilum, and wherein the first, second, and third RPA oligonucleotide pairs do not substantially cross react with the other species of Anaplasma;
a plurality of reaction chambers, each for performing a recombinase polymerase amplification reaction, wherein each reaction chamber comprises a reagent composition comprising a recombinase, a polymerase, and a single-stranded DNA binding protein; and
a plurality of nucleic acid lateral flow assay devices.

16. The system of claim 15, further comprising at least one collection device.

17. A screening method, comprising:
obtaining a mammalian sample suspected of containing at least one species of Anaplasma;
performing at least one RPA reaction with at least one of the RPA oligonucleotide pairs of the kit of claim 1; and
determining if the species of Anaplasma for which the RPA oligonucleotide pair is specific is present in the sample based on the result of the at least one RPA reaction.

18. The screening method of claim 17, further comprising the step of:
performing at least a second RPA reaction with at least another of the RPA oligonucleotide pairs of the kit of claim 1; and
determining if the species of Anaplasma for which the RPA oligonucleotide pair is specific is present in the sample based on the result of the second RPA reaction.

19. The screening method of claim 18, further comprising the step of:
performing at least a third RPA reaction with at least another of the RPA oligonucleotide pairs of the kit of any of claim 1; and
determining if the species of Anaplasma for which the RPA oligonucleotide pair is specific is present in the sample based on the result of the third RPA reaction.

20. The screening method of claim 17, wherein the step of performing each of the RPA reactions comprises the steps of:
combining the mammalian sample with the RPA oligonucleotide pair and an RPA reagent composition to provide a mixture and incubating the mixture under conditions that allow amplification to occur, wherein the RPA reagent composition comprises a recombinase, a polymerase, and a single-stranded DNA binding protein; and
contacting the incubated mixture with a nucleic acid lateral flow assay device; and
detecting the RPA product via the nucleic acid lateral flow assay device.
